(19) United States
(12) Patent Application Publication
(10) Pub. No.: US 2025/0195885 A1
(43) Pub. Date: Jun. 19, 2025

(54) AUTOMATIC DETERMINATION OF STIMULATION SETTINGS BASED ON SIMILARITY METRICS

(71) Applicant: Boston Scientific Neuromodulation Corporation, Valencia, CA (US)

(72) Inventors: Leon Mauricio Juarez Paz, Berlin (DE); Hemant Bokil, Cambridge, MA (US)

(21) Appl. No.: 18/974,449

(22) Filed: Dec. 9, 2024

Related U.S. Application Data
(60) Provisional application No. 63/610,307, filed on Dec. 14, 2023.

(51) Int. Cl. A61N 1/36

(52) U.S. Cl. CPC A61N 1/36031; A61N 1/36034

(57) ABSTRACT

A system and method for automated neurostimulation programming uses machine learning to determine target stimulation or discard suboptimal parameters based on actual or anticipated clinical effects. The system includes processors to generate a database of previously acquired lead and/or brain images, tested stimulation settings and associated clinical effects. A region of interest surrounding at least one stimulation lead is identified. Stimulation settings linked to specific clinical effects, including beneficial or detrimental effects, are determined for the region of interest. A trained machine learning model identifies desirable, undesirable, target, or other stimulation parameter values for a new patient by classifying or regressing raw imaging data from the region of interest. The model is trained to recommend settings that produce desired clinical effects. An output provides the target stimulation parameter values predicted to maximize therapeutic benefit and minimize side effects. This enables automated programming of neurostimulation devices through data-driven machine learning based on clinical outcomes.

### CLAIM OF PRIORITY

This application claims the benefit of U.S. Provisional Application No. 63/610,307, filed on Dec. 14, 2023, which is hereby incorporated by reference in its entirety.

### TECHNICAL FIELD

This document relates generally to medical systems, and more particularly, but not by way of limitation, to systems, devices, machine-readable media, and methods for using machine learning based automated neurostimulation programming and automatic determination of stimulation settings based on similarity metrics for deep brain stimulation, electrical stimulation in the treatment, research, and/or management of pain and other conditions.

### BACKGROUND

Chronic pain, such as pain present most of the time for a period of six months or longer during the prior year, is a highly pervasive complaint and consistently associated with mental, physical, and/or psychological illnesses. Chronic pain can originate with a trauma, injury or infection, or there can be an ongoing cause of pain. Chronic pain can also present in the absence of any past injury or evidence of body damage. Common chronic pain can include headache, low back pain, cancer pain, arthritis pain, neurogenic pain (pain resulting from damage to the peripheral nerves or to the central nervous system), somatic pain, psychogenic pain (pain not due to past disease or injury or any visible sign of damage inside or outside the nervous system), or other types of pain. However, chronic pain is far more than the existence of a physical sensation or feeling of pain. Chronic pain is not just a number rating on a scale but can be a life-consuming change to every moment of every day for a pain patient. Chronic pain has a significant impact on a person's quality of life, affecting not only physical health but also emotional and social well-being. Chronic pain can affect a patient's physical limitations, cause emotional and psychological distress, increase social isolation, bring about financial strain, decrease quality of life, and much more.

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

The neurostimulation energy may be delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Many current neurostimulation systems are programmed to deliver periodic pulses with one or a few uniform waveforms continuously or in bursts. However, neural signals may include more sophisticated patterns to communicate various types of information, including sensations of pain, pressure, temperature, etc. The nervous system may interpret an artificial stimulation with a simple pattern of stimuli as an unnatural phenomenon and respond with an unintended and undesirable sensation and/or movement. For example, some neurostimulation therapies are known to cause paresthesia and/or vibration of non-targeted tissue or organ.

### SUMMARY

Various embodiments of the present subject matter categorize a pain patient into one of several therapeutic categories according to parameterizing techniques for sub-perception therapy and multi-sensor paresthesia therapy, provide patient treatment changes remotely, monitor parameters, or a combination thereof.

Described implementations of the subject matter can include one or more features, alone or in combination as illustrated below by way of example.

Example 1 is a system for automated determination of stimulation parameters through analysis of patient medical imaging data, the system comprising: one or more processors; and one or more memory storing instructions, which when executed by the one or more processors, cause the one or more processors to perform operations that: store, in one or more databases, multimodal medical imaging data for a plurality of patients using stimulation therapy, the multimodal medical imaging data including voxel intensity data; access, from the one or more databases, the multimodal medical imaging data; calculate one or more similarity metrics between each patient of the plurality of patients directly from a native space including the multimodal medical imaging data; use the one or more similarity metrics to cluster the plurality of patients into a phenotypic group based on the extracted biomarkers; access, from the one or more databases, therapeutic outcomes achieved for each patient of the plurality of patients, including applied stimulation parameter settings associated with the therapeutic outcomes; determine a target stimulation parameter setting for a new patient predicted to achieve beneficial therapeutic effects by identifying one or more phenotypically similar groups based on medical imaging data biomarkers associated with the new patient; and generate an output including the target stimulation parameter setting for the new patient.

In Example 2, the subject matter of Example 1 includes, wherein the multimodal medical imaging data comprises voxel intensity values from imaging scans, without requiring registration to a standardized atlas or template.

In Example 3, the subject matter of Examples 1-2 includes, wherein the one or more similarity metrics include a database of medical imaging data from the plurality of patients corresponding to therapeutic outcomes based on previously applied neurostimulation parameters, and wherein the one or more similarity metrics are calculated directly from the multimodal medical imaging data without requiring spatial normalization or warping to a common coordinate system.

In Example 4, the subject matter of Examples 1-3 includes, wherein determining the target stimulation parameter setting for the new patient is performed without stimulation field modeling.

In Example 5, the subject matter of Examples 1-4 includes, wherein the instructions cause the one or more processors to further perform the operations that: analyze the voxel intensity data to extract biomarkers predictive of therapeutic responses for each patient of the plurality of patients; and calculate the one or more similarity metrics using a clustering algorithm or deep neural network.

In Example 6, the subject matter of Examples 1-5 includes, wherein the instructions cause the one or more processors to further perform the operations that: store a database of medical imaging data from the plurality of patients and corresponding therapeutic outcomes for previously applied neurostimulation parameters.

In Example 7, the subject matter of Example 6 includes, wherein the instructions cause the one or more processors to further perform the operations that: access the database to identify a subset of patients from the plurality of patients similar to the new patient; and determine the target stimulation parameter setting based on corresponding outcomes from the subset of patients.

In Example 8, the subject matter of Examples 1-7 includes, wherein the one or more similarity metrics are calculated based on the multimodal medical imaging data including structural imaging data and functional imaging data.

In Example 9, the subject matter of Examples 1-8 includes, wherein the target stimulation parameter setting comprise at least one of an amplitude, a pulse width, a stimulation frequency, an electrode contact configuration, a pulse type, a pattern type, a sequence, a duty cycle, or an electrode fractionalization.

In Example 10, the subject matter of Examples 1-9 includes, wherein the instructions cause the one or more processors to further perform the operations that: calculate the one or more similarity metrics using a deep neural network trained on the multimodal medical imaging data.

In Example 11, the subject matter of Example 10 includes, wherein the deep neural network comprises a convolutional neural network and/or recurrent neural network.

In Example 12, the subject matter of Examples 1-11 includes, wherein the instructions cause the one or more processors to further perform the operations that: generate a user interface to be displayed, the user interface configured to visualize the target stimulation parameter setting.

In Example 13, the subject matter of Examples 1-12 includes, wherein the instructions cause the one or more processors to further perform the operations that: calculate the one or more similarity metrics using a clustering algorithm.

In Example 14, the subject matter of Examples 1-13 includes, wherein the instructions cause the one or more processors to further perform the operations that: provide a user interface configured to receive user input for adjusting the target stimulation parameter setting.

In Example 15, the subject matter of Examples 1-14 includes, wherein the instructions cause the one or more processors to further perform the operations that: recalculate, in an iterative manner, the target stimulation parameter setting based on updated therapeutic outcomes for the new patient.

Example 16 is a method for automated determination of stimulation parameters through analysis of patient medical imaging data, the method comprising: storing, in one or more databases, multimodal medical imaging data for a plurality of patients using stimulation therapy, the multimodal medical imaging data including voxel intensity data; accessing, from the one or more databases, the multimodal medical imaging data; calculating one or more similarity metrics between each patient of the plurality of patients directly from a native space including the multimodal medical imaging data; using the one or more similarity metrics to cluster the plurality of patients into a phenotypic group based on the extracted biomarkers; accessing, from the one or more databases, therapeutic outcomes achieved for each patient of the plurality of patients, including applied stimulation parameter settings associated with the therapeutic outcomes; determining a target stimulation parameter setting for a new patient predicted to achieve beneficial therapeutic effects by identifying one or more phenotypically similar groups based on medical imaging data biomarkers associated with the new patient; and generating an output including the target stimulation parameter setting for the new patient.

In Example 17, the subject matter of Example 16 includes, wherein the multimodal medical imaging data comprises voxel intensity values from imaging scans, without requiring registration to a standardized atlas or template.

In Example 18, the subject matter of Examples 16-17 includes, wherein the one or more similarity metrics include a database of medical imaging data from the plurality of patients corresponding to therapeutic outcomes based on previously applied neurostimulation parameters, and wherein the one or more similarity metrics are calculated directly from the multimodal medical imaging data without requiring spatial normalization or warping to a common coordinate system.

In Example 19, the subject matter of Examples 16-18 includes, wherein determining the target stimulation parameter setting for the new patient is performed without stimulation field modeling.

In Example 20, the subject matter of Examples 16-19 includes, analyzing the voxel intensity data to extract biomarkers predictive of therapeutic responses for each patient of the plurality of patients; and calculating the one or more similarity metrics using a deep neural network.

In Example 21, the subject matter of Examples 16-20 includes, storing, in the one or more databases, medical imaging data from the plurality of patients and corresponding therapeutic outcomes for previously applied neurostimulation parameters.

In Example 22, the subject matter of Example 21 includes, accessing the one or more databases to identify a subset of patients from the plurality of patients similar to the new patient; and determining the target stimulation parameter setting based on corresponding outcomes from the subset of patients.

In Example 23, the subject matter of Examples 16-22 includes, wherein the one or more similarity metrics are calculated based on the multimodal medical imaging data including structural imaging data and functional imaging data.

In Example 24, the subject matter of Examples 16-23 includes, wherein the target stimulation parameter setting comprise at least one of an amplitude, a pulse width, a stimulation frequency, an electrode contact configuration, a pulse type, a pattern type, a sequence, a duty cycle, or an electrode fractionalization.

In Example 25, the subject matter of Examples 16-24 includes, calculating the one or more similarity metrics using a deep neural network trained on the multimodal medical imaging data.

In Example 26, the subject matter of Example 25 includes, wherein the deep neural network comprises a convolutional neural network and/or recurrent neural network.

In Example 27, the subject matter of Examples 16-26 includes, generating a user interface to be displayed, the user interface configured to display a visualization of the target stimulation parameter setting.

In Example 28, the subject matter of Examples 16-27 includes, providing a user interface configured to receive user input for adjusting the target stimulation parameter setting.

Example 29 is a machine-storage medium embodying instructions that, when executed by a machine, cause the machine to perform operations comprising: storing, in one or more databases, multimodal medical imaging data for a plurality of patients using stimulation therapy, the multimodal medical imaging data including voxel intensity data; accessing, from the one or more databases, the multimodal medical imaging data; calculating one or more similarity metrics between each patient of the plurality of patients directly from a native space including the multimodal medical imaging data; using the one or more similarity metrics to cluster the plurality of patients into a phenotypic group based on the extracted biomarkers; accessing, from the one or more databases, therapeutic outcomes achieved for each patient of the plurality of patients, including applied stimulation parameter settings associated with the therapeutic outcomes; determining a target stimulation parameter setting for a new patient predicted to achieve beneficial therapeutic effects by identifying one or more phenotypically similar groups based on medical imaging data biomarkers associated with the new patient; and generating an output including the target stimulation parameter setting for the new patient.

In Example 30, the subject matter of Example 29 includes, accessing the one or more databases to identify a subset of patients from the plurality of patients similar to the new patient; and determine the target stimulation parameter setting based on corresponding outcomes from the subset of patients.

In Example 31, the subject matter of Examples 29-30 includes, wherein the one or more similarity metrics include a database of medical imaging data from the plurality of patients corresponding to therapeutic outcomes based on previously applied neurostimulation parameters, and wherein the one or more similarity metrics are calculated based on the multimodal medical imaging data including structural imaging data and functional imaging data.

In Example 32, the subject matter of Examples 29-31 includes, wherein the target stimulation parameter setting comprise at least one of an amplitude, a pulse width, a stimulation frequency, an electrode contact configuration, a pulse type, a pattern type, a sequence, a duty cycle, or an electrode fractionalization.

In Example 33, the subject matter of Examples 29-32 includes, analyzing the voxel intensity data to extract biomarkers predictive of therapeutic responses for each patient of the plurality of patients; and calculating the one or more similarity metrics using a deep neural network trained on the multimodal medical imaging data.

In Example 34, the subject matter of Examples 29-33 includes, generating a user interface to be displayed, the user interface configured to visualize the target stimulation parameter setting.

Example 35 is a system for automated determination of stimulation parameters through analysis of patient medical imaging data, the system comprising: one or more processors; and one or more memory storing instructions, which when executed by the one or more processors, cause the one or more processors to perform operations that: store, in one or more databases, multimodal medical imaging data for a plurality of patients using stimulation therapy, the multimodal medical imaging data including voxel intensity data; access, from the one or more databases, the multimodal medical imaging data; calculate one or more similarity metrics between each patient of the plurality of patients directly from a native space including the multimodal medical imaging data; use the one or more similarity metrics to cluster the plurality of patients into a phenotypic group based on the extracted biomarkers; access, from the one or more databases, therapeutic outcomes achieved for each patient of the plurality of patients, including applied stimulation parameter settings associated with the therapeutic outcomes; determine a target stimulation parameter setting for a new patient predicted to achieve beneficial therapeutic effects by identifying one or more phenotypically similar groups based on medical imaging data biomarkers associated with the new patient; and generate output including the target stimulation parameter setting for the new patient.

Example 36 is at least one machine-readable medium including instructions that, when executed by processing circuitry, cause the processing circuitry to perform operations to implement of any of Examples 1-35.

Example 37 is an apparatus comprising means to implement of any of Examples 1-35.

Example 38 is a system to implement of any of Examples 1-35.

Example 39 is a method to implement of any of Examples 1-35.

This Summary is an overview of some of the teachings of the present application and not intended to be an exclusive or exhaustive treatment of the present subject matter. Further details about the present subject matter are found in the detailed description, figures, and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE SEVERAL VIEWS OF THE DRAWINGS

This document relates generally to medical systems, and more particularly, but not by way of limitation, to systems, devices, machine-readable media, and methods for using healthcare-related data to improve patient monitoring, automatically determining stimulation settings, and/or providing treatment options for implanted electrical stimulation for treatment according to machine learning based automated programming. Various embodiments are illustrated by way of example in the figures of the accompanying drawings. Such embodiments are demonstrative and not intended to be exhaustive or exclusive embodiments of the present subject matter.

FIG. 1A illustrates, by way of example and not limitation, a neuromodulation system implemented in a spinal cord stimulation system or a deep brain stimulation system, in accordance with one embodiment.

FIG. 1B illustrates, by way of example, a neuromodulation system of implemented in a deep brain stimulation system to automatically identify DBS parameter settings based on imaging data of a plurality of patients, in accordance with one embodiment.

FIGS. 2A-2C illustrate, by way of example, a series of radiographic imaging images identifying regions of interest in a patient to determine stimulation setting parameters, in accordance with one embodiment.

FIGS. 3A-3B illustrate, by way of example, medical imaging scans in a variety of bodily orientations identifying similarities based on voxels corresponding to a brain object around a lead, in accordance with example embodiments.

FIG. 4 illustrates, by way of example and not limitation, two graphs depicting data on patients with similarities in order to automatically predict stimulation settings, in accordance with one embodiment.

FIGS. 5A-5B illustrate, by way of example, diagrams depicting empirical determination of amplitude and fractionalization values used to constrain a search space for a particular patient of interest, in accordance with example embodiments.

FIGS. 6A-6C illustrate, by way of example, a variety of block diagrams showing different targets, fields of view, and lead-target relationships from imaging data used to train machine learning model(s), in accordance with example embodiments.

FIG. 7 illustrates, by way of example, a block diagram depicting a data fusion module used to train a network on medical imaging data and/or other data to automatically predict stimulation parameters, in accordance with one embodiment.

FIG. 8 illustrates, by way of example, an embodiment of a neurostimulation system communicatively coupled to various databases via a communication system, in accordance with one embodiment.

FIG. 9 is a flowchart illustrating a method of automatically determining stimulation settings based on similarity metrics, in accordance with one embodiment.

FIG. 10 is a flowchart illustrating a method of determining stimulation settings based on similarity metrics, in accordance with one embodiment.

FIG. 11 is a flowchart illustrating a method of predicting stimulation settings utilizing information from voxel intensities from imaging data in areas adjacent to implanted leads from a patient's native space imaging, in accordance with one embodiment.

FIG. 12A illustrates, by way of example, a block diagram of an embodiment of a system (e.g., a computing system) implementing neurostimulation programming circuitry to cause programming of an implantable electrical neurostimulation device, in accordance with one embodiment.

FIG. 12B illustrates, by way of example, a block diagram of an embodiment of a system for performing patient data analysis in connection with programming operations, in accordance with one embodiment.

FIG. 13A illustrates, by way of example and not limitation, a patient system, and examples of devices that may make up components of the patient system, in accordance with one embodiment.

FIG. 13B illustrates a patient system and examples of devices that may make up components of the patient system, in accordance with one embodiment.

FIG. 14 illustrates, by way of example, an embodiment of a programming system and data analysis system for use with a neurostimulation system, such as the implantable neuromodulation system of FIG. 1A, in accordance with one embodiment.

FIG. 15 illustrates, by way of example, an embodiment of data interactions among a data analysis computing system and clinician and patient interaction computing devices, for determining similarity metrics between patients, in accordance with one embodiment.

FIG. 16 illustrates, by way of example, an embodiment of a data processing flow for affecting the neurostimulation treatment of a human patient, based on text, image, and/or device data processing to identify similarity metrics, in accordance with one embodiment.

FIG. 17 illustrates a machine-learning pipeline, in accordance with one embodiment.

FIG. 18 illustrates training and use of a machine-learning program, in accordance with one embodiment.

FIG. 19 is a block diagram illustrating a machine in the example form of a computer system, within which a set or sequence of instructions can be executed to cause the machine to perform any one of the methodologies discussed herein, in accordance with one embodiment.

### DETAILED DESCRIPTION

The following detailed description of the present subject matter refers to the accompanying drawings which show, by way of illustration, specific aspects and embodiments in which the present subject matter can be practiced. These embodiments are described in sufficient detail to enable those skilled in the art to practice the present subject matter. Other embodiments can be utilized, and structural, logical, and electrical changes can be made without departing from the scope of the present subject matter. References to “an,” “one,” or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

### Overview

Examples of neurostimulation include Deep Brain Stimulation (DBS), Spinal Cord Stimulation (SCS), Peripheral Nerve Stimulation (PNS), Functional Electrical Stimulation (FES), and other forms of implantable, transcutaneous, or wearable stimulation devices. Neurostimulation systems have been applied to deliver such a therapy. The neurostimulator delivers neurostimulation energy through one or more electrodes or paddles placed on or near a target site in the nervous system. An external programming device (e.g., a remote, application, mobile device, etc.) is used to program the stimulator with stimulation parameters controlling the delivery of the stimulation energy. The apparatus and methods recited in example embodiments of the present disclosure can apply to medical, veterinary, and/or engineering devices that are used as a component of numerous different types of stimulation systems; this may include all the different variations of DBS, SCS, PNS, FES, etc. that fall within the general term “stimulator” or “stimulation device” as understood by a person having ordinary skill in the art. For ease of description and understanding, examples presented herein will refer to a “DBS” as an example most generally understood in the field of neurostimulation; however, all such examples will not be recited, for brevity. However, it is to be understood that the while the invention lends itself well to applications in DBS and SCS, the invention, in its broadest aspects, may not be so limited. Rather, the invention may be used with any type of implantable electrical circuitry used to stimulate tissue. For example, the present inventions may be used as part of a pacemaker, a defibrillator, a cochlear stimulator, a retinal stimulator, a stimulator configured to produce coordinated limb movement, a cortical stimulator, a peripheral nerve stimulator, a microstimulator, or in any other neural stimulator configured to treat urinary incontinence, sleep apnea, shoulder subluxation, headache, Parkinson's Disease, neurological condition, or psychiatrist disorder that is treatable by neurostimulation etc.

In one example, the neurostimulation energy is delivered in the form of electrical neurostimulation pulses. The delivery is controlled using stimulation parameters that specify spatial (e.g., where to stimulate), temporal (e.g., when to stimulate), and informational (e.g., patterns of pulses directing the nervous system to respond as desired) aspects of a pattern of neurostimulation pulses. The human nervous systems use neural signals having sophisticated patterns to communicate various types of information, including sensations of pain, pressure, temperature, etc. It may interpret an artificial stimulation with a simple pattern of stimuli as an unnatural phenomenon and respond with an unintended and undesirable sensation and/or movement. Also, as the condition of the patient may change while receiving a neurostimulation therapy, the pattern of neurostimulation pulses applied to the patient may need to be changed to maintain efficacy of the therapy (e.g., beneficial clinical effects) while minimizing the unintended and undesirable sensation and/or movement (e.g., detrimental clinical effects). While modern electronics can accommodate the need for generating sophisticated pulse patterns that emulate natural patterns of neural signals observed in the human body, the capability of a neurostimulation system depends on its post-manufacturing programmability to a great extent. For example, a sophisticated pulse pattern may only benefit a patient when it is customized for that patient and updated timely in response to changes in the patient's conditions and needs. This makes programming of a stimulation device for a patient a challenging task.

In accordance with a first aspect of the present inventions, a method for performing automatic determination of stimulation settings based on similarity metrics is provided. The method includes using previously gathered and organized patient data and/or artificially generated patient data to calculate similarity metrics between patients and use these similarity metrics individually or in combination to determine stimulation parameters values with a higher likelihood of beneficial clinical effects and/or detrimental clinical effects.

In some examples of the first aspect of the present inventions, deep brain stimulation (DBS) settings optimization is performed. DBS settings optimization can usually involve empirically determining values for parameters such as pulse width, frequency, electrode fractionalization, current amplitude, and the like. Such parameter values can lead either to beneficial clinical effects, undesired or intolerable side effects, detrimental, or no change in clinical effects. The empirical determination of these parameter values is usually time consuming and can cause potential discomfort to the patient. In addition to timing, typically, analysis of imaging data involves warping or registering each patient's images to a standard atlas or template space to allow comparisons and transfer of knowledge. This requires using warping tools and anatomical segmentation software to map the images into a common coordinate system. Prior attempts to solve for optimal DBS setting optimization need specialized warping and/or segmentation software tools, such as BRAINLAB QUENTRY® or BRAINLAB ELEMENTS®.

Existing approaches of chronic pain treatment include conventional in-person visits to a clinic, telehealth appointments, or other scheduled doctor visits, which require person-to-person pain assessments in a clinical setting. Often, a patient will end up needing to provide detailed feedback to a clinician before a treatment issue can be identified and changes can be implemented to a neurostimulation treatment; this can take weeks or even months from onset to triage to treatment implementation. Existing approaches of neurological disorder treatments (e.g., Parkinson's Disease, etc.) can include deep brain stimulation (DBS) of the thalamus, STN (subthalamic nucleus), or GPi (Globus pallidus) is often used to improve symptoms of neurological disorders. However, adjustment of DBS by a neurologist is traditionally done through a serial process where the neurologist makes a program adjustment, observes a certain symptom (e.g., tremor, arm rigidity), task (e.g., finger-tapping, rapidly alternating movement), or side effect (e.g., dysarthria, muscle twitches), and then makes further adjustments. This is time-consuming and may fail to optimize the stimulation settings across all symptoms in all areas of the body.

Prior approaches for a neurostimulation system depend on its post-manufacturing programmability to a great extent. One limiting factor for applications of neurostimulation therapies is that, even if a number of advanced programs can be applied by a neurostimulation device, there is often a delay for implementing new or improved neurostimulation treatments. Such a delay can be due to the infrequency of care provided by a clinician or other medical professional who oversees the treatment, and a lack of clear information regarding the results of the treatment (e.g., whether or not sub-perception therapies are providing relief to the patient). Various approaches for neurostimulation programming and customization have attempted more dynamic forms of open-loop and closed-loop programing, to allow new neurostimulation parameters or programs to be introduced, deployed, tested, and adjusted by a clinician, the subject patient, a software program, model, or the like. Although some neurostimulation devices provide the capability to enable a patient to switch between programs or change the level of a certain stimulation effect, it is often unclear whether such changes (or, which changes) are beneficial to a patient and result in improvement to the patient's medical condition.

In such prior existing approaches of DBS predictive programming and aggregation, technologies aggregate data by transforming patient-specific data into a common space for aggregation to develop optimal and suboptimal stimulation targets (e.g., targets eliciting maximal and minimal therapeutic effects, loci exhibiting heightened and attenuated treatment sensitivity, foci demonstrating maximal and minimal therapeutic activation, etc.). Prior technologies using DBS programming and aggregation approaches require the use of a common space program in order to transform patient specific data into the common space to predict or develop sweet and sour spots of treatment. Such prior approaches may face multiple issues/challenges. For example, using a common space for aggregation may represent patient brains using a Mean+Noise model, and treats surgical technique overlap as equivalent to physiological overlap. For example, three stimulation field models (SFMs) in a picture all give five points of improvement, but even small intersection regions are considered better than the others. In addition, using a common space relies on simplifying assumptions inherent in stimulation field modeling (SFM) (e.g., single fiber diameter, homogenous brain tissue, etc.).

Example embodiments of systems, methods, machine-readable medium, software, and artificial intelligence provide for improved approaches for automatic determination of stimulation settings based on similarity metrics, such as calculation of similarity metrics based on or relying on imaging data while avoiding the need of warping tools or anatomical segmentation software. Example embodiments of the present disclosure provide for systems to generate database(s) of previously tested stimulation settings and their corresponding clinical effect(s), which are used to determine stimulation settings leading to specific clinical effects (e.g., beneficial effects or side effects (negative effects)). Examples for generating a database and determining stimulation settings can run separately or in parallel to determine similarity between patients using a single metric, multiple metrics, or a combination of metrics and other patient health data.

According to examples, once a database is generated and the stimulation settings are determined, embodiments of the system can cluster stimulation settings from similar patients and provide an output for the user. According to example embodiments of the present disclosure, the user can be a patient, clinician, doctor, caregiver, device representative, software application, or the like. The output can include, for example, suggestions of stimulation settings that are encouraged or to be avoided, based on cluster features (e.g., size, density, etc.). The output can be provided via one or more graphical user interface(s) (GUI) for data inputs and outputs displayed to the user via an application (e.g., a smartphone, application, computer, remote, etc.).

Example embodiments of the present disclosure improve upon existing models and overcome such current technical challenges by providing a different or additional option for automatized DBS programming algorithms to calculate similarity metric(s) between imaging data from different patients without needing to warp the images into a common atlas space. According to examples, the similarity metric(s) to be calculated can be based on voxel intensities in imaging scans (e.g., MRI scans, CT scans, etc.) that can provide strategic independence from current technologies that require automatic segmentation software. The similarity metric(s) can further be based on anatomical data without the need of software to perform patients' aggregation into a common space. In other words, example embodiments for calculating a similarity metric avoids the need for specialized warping and/or segmentation software tools by directly comparing the imaging data between patients in their native space, without warping to an atlas. By calculating similarities from imaging data in native patient space rather than a warped atlas space, the invention provides greater flexibility and reduces reliance on specific software tools. For example, the similarity metric can be calculated without the preprocessing step of atlas registration and anatomical segmentation.

Example embodiments of the present disclosure include a method of employing previously gathered and organized data (e.g., actual patient data, simulated patient data, etc.) to calculate one or more similarity metrics between patients and using the one or more similarity metrics, individually or combined, to determine stimulation parameter values with a higher likelihood of real and/or anticipated (e.g., calculated, determined, presumed, inferred, etc.) clinical effects, such as beneficial clinical effect(s), detrimental clinical effect(s), or providing no significant clinical effect(s). One example advantage of the proposed technique is the ability to calculate similarity metrics directly from patients' native imaging space, bypassing the typical steps of warping or registering each patient's images to a standardized atlas or template. Avoiding atlas warping preserves patient-specific anatomical nuances that may be distorted during spatial normalization and avoid introduction of inevitable errors and biases during such process. Furthermore, calculating similarities in native space eliminates the need for time-intensive image registration and warping, reduces reliance on specialized atlas software tools, and enables fully automated analysis. The algorithms employed can be robust to inter-patient anatomical variability in native space.

In some examples, one of the proposed similarity metrics relies on imaging data. The imaging data, as used herein in the context of the present invention, can include three-dimensional voxel data from medical imaging modalities including, for example, magnetic resonance imaging (MRI), computed tomography (CT), positron emission tomography (PET) scans or the like, which generate digital representations of a patient's anatomy by capturing high-resolution views of soft tissue contrast, spatial relationships, shape characteristics, and functional activities non-invasively. This imaging data consists of arrays of voxel intensity values at regular grid coordinates corresponding to specific locations within the target anatomy. Specialized image processing and analysis techniques can extract meaningful information from the data, such as identifying anatomical structures and physiological biomarkers predictive of disease states or therapeutic responses. According to some example embodiments, examples of the present disclosure can include different patient settings and attributes, or combinations of processes; for example, some examples do not require identification of biomarkers directly in the imaging (e.g., a first patient imaging set may use other markers). According to some examples, the system can eliminate the need to identify biomarkers in imaging for use with atlas, registration-free, or other methods and the like. It will be understood by those having ordinary skill in the art that different combinations of parameters and/or settings may be used.

Multimodal imaging combines complementary modalities like MRI, CT, PET, etc. to provide improved insights compared to any single modality alone. For example, embodiments of the present inventions include imaging data of a patient's brain and surrounding anatomy enables identification of structural and functional targets to guide deep brain stimulation therapy. Machine learning algorithms can then analyze patterns in multimodal imaging data to determine optimal patient-specific treatment parameters tailored to an individual's unique anatomy and disease biomarkers. As will be understood by those having skill in the art, imaging scans can provide data of other portions of a patient's body and surrounding anatomy, such as a patient's spinal cord; however, for simplicity in explanation, the brain will be used in example embodiments and MRI data will be used as the example imaging.

The optimal value, as used herein in the context of the present invention, refers to the therapeutic, effective, efficacious, beneficial, preferable, favorable, advantageous, desired, ideal, optimized, target, intended, maximized, minimized, predetermined, preselected, calculated, derived, or estimated value that provides the greatest medicinal benefit, achieves the intended effect, produces the desired result, offers positive advantages, provides superiority in performance, represents the most favorable option, has been adjusted to reach the best achievable performance, fulfills the identified goal, increases to the highest amount, decreases to the lowest amount, is decided upon in advance, is chosen beforehand, is determined mathematically, is obtained through computation, or is approximated based on available data analyzed by the invention. The optimal value enhances the invention's objectives through improved treatment outcomes, advantageous attributes aligned with performance goals, planned objectives, and data-driven quantification of favorable parameter values tailored to the context and purpose of the particular application. Determining the optimal value enables the invention to maximize therapeutic efficacy for the patient. Conversely, a sub-optimal value or minimal value refers to the opposite of optimal value, referring to the lowest possible value that may still be acceptable but is far from ideal, that minimizes the desired outcome, or has a lower likelihood of beneficial clinical effect. The sub-optimal value can be an important value to show detrimental clinical effects.

In accordance with a second aspect of the present inventions, a method for performing machine learning based automated neurostimulation programming is provided. Example embodiments of systems, methods, machine-readable medium, software, and artificial intelligence provide for using a multi-layer neural network, for example, or deep structured machine learning model (e.g., deep neural networks (DNN)) to predict the stimulation parameters without recourse to any aggregation or common space analysis. In addition, examples of the present disclosure may not need to use SFMs either. Additional examples, when not employing automated programming, the neurostimulation programming can use other programming types, such as clinical decision support or the like.

Example embodiments include implementing (e.g., employing, utilizing, etc.) deep learning approaches in targeting to provide patient specific segmentation of various nuclei relevant to DBS. Embodiments directly utilize information available from the voxel intensities (e.g., from imaging data) in the areas immediately adjacent to the implanted lead, directly from the patient's native space imaging, and/or without any normalization to a standard or atlas space. For example, the areas immediately adjacent (e.g., a few millimeters, tens of voxels in each direction, or other measurement) to the implanted lead can include looking at the anatomical regions in close vicinity to the targeted region in order to analyze those (e.g., similarity, etc.), such as within a few tens of voxels in each direction, for example and not limitation. In other examples, one can look at the entire brain, for example, and not just regions adjacent.

Such examples provide for improved predictive programing and aggregation of patients' data. For example, when three patients are showing similar improvements due to the DBS, that improvement is highly likely happening because DBS is doing something similar (e.g., having similar clinical effects) to their brains. In so far as the effect of DBS begins at the local level (e.g., a region around the lead), there must be something (e.g., a variable) in common in the local region around the lead between these patients. As such, it is possible to capture that common variable without recourse to a common atlas, space, or SFM.

In addition, information available from the structural components of the neuroanatomical areas relevant to the DBS (e.g., most prominently those in immediate vicinity to the lead(s)) are integral to all processes that form brain atlases (e.g., such as the standard brain). For example, SFMs are modeled based on processes that synthesize these structural features, therefore, a system that can directly use a machine to learn structural properties of these brain areas can provide necessary or wanted information to predict outcomes, while staying clear from the simplifying assumptions and/or noise inherent in the data aggregation processes.

According to example embodiments of the present disclosure, using a machine learning algorithm, deep neural network, clustering algorithm, or the like to identify a plurality of patients with an implanted neurostimulation device in a variety of anatomical areas (e.g., different regions of the patient's brains), where each of the plurality of patients or the majority of the plurality of patients is responding to the neurostimulation in the same or similar way as each of the other patients, the present disclosure can identify that a variable exists amongst all or most of the patients that is a common variable (e.g., commonality in a region around a lead, commonality in the patients' physiology causing the same response, etc.). The example embodiments can use machine learning to automatically detect (e.g., predict) commonalities in patients without requiring a process of registering the data to a common space. For example, a DNN can be used to identify (e.g., infer) commonalities between different patients, different datasets, different imaging data, or combinations thereof.

According to example embodiments of the present disclosure, based on machine learning models identification of similarities and differences among datasets, the output of said machine learning models can identify one or more similarity or dissimilarity metrics among patients. In some examples, a subset of patient data can be used to identify inferences that make a patient of interest (e.g., a current patient) similar to other identified patients in a subset of a plurality of patients. For example, the subset of patients whose brains are the most similar and linear/non-linear combinations of those brains can be used to construct the prediction that is most similar to the test (e.g., de novo patient).

According to example embodiments, a patient's entire brain scan can be provided in data imaging datasets; however, an entire brain incorporates vast amounts of anatomical differences between patients that may be too broad to identify one or more similarity or dissimilarity metrics among patients. Accordingly, embodiments identify one or more regions (e.g., regions of interest) around one or more leads to identify (e.g., find) the common features of that region around the lead in the imaging (e.g., radiographic scans such as MRIs). Based on the output of the machine learning models, where the output identifies one or more commonalities, example embodiments of the present disclosure further infer where a patient should be stimulated and/or with what amplitude, for example, the patient should be stimulated.

In addition to deep neural networks, less ‘blind’ (supervised) machine learning methods could also be used to determine similarities between patients and group them into phenotypic clusters. For example, clustering algorithms could be leveraged along with manually engineered feature vectors that characterize relevant attributes of each patient. These features could include, by way of example and not limitation, demographic information, specific disease characteristics, anatomical traits from medical imaging, genetics, symptom profiles, and more. By using predefined features that are hypothesized to relate to therapeutic outcomes, patients can be clustered into groups that share common traits associated with those engineered features. This allows patients with similar features related to the predefined vectors to be clustered together, while patients with different features would fall into separate clusters. The result is phenotypic stratification of patients based on hand-selected variables anticipated to impact optimal stimulation parameters and outcomes. This provides an alternative to deep learning techniques that automatically learn the relevant features directly from the data. The predefined features support interpretability, while clustering allows discovering data-driven patient groups that can inform stimulation programming decisions. In other words, rather than relying solely or primarily on a black-box machine learning models to discover patterns, some example embodiments can include an approach using predefined feature vectors to represent specific aspects of patients' disease state, anatomy, outcomes, etc. For example, feature vectors could explicitly encode information like location of the implanted DBS lead relative to anatomical landmarks, volume/shape of the patient's subthalamic nucleus, presence/absence of certain symptoms or comorbidities, change in motor scores after programming, or the like. These hand-engineered feature vectors can then be used as input to a clustering algorithm, like k-means, to group patients based on similarities along these predefined dimensions.

Additional example embodiments can include patient populations receiving neurostimulation therapy can exhibit diverse phenotypic characteristics including differences in anatomy, physiology, genetics, biomarkers, symptomatology, disease progression patterns, or the like. Rather than a one-size-fits-all approach, the present invention leverages phenotypic stratification, which involves identifying and analyzing phenotypic differences between patients in order to separate the population into more homogenous subgroups. For example, patients could be stratified based on imaging biomarkers, genetics, symptom profiles, anatomical traits, demographics, comorbidities, or other measures. This allows tailoring selection of optimal neurostimulation parameters and treatment decisions to specific patient subgroups. By grouping patients exhibiting similar therapeutic responses and disease phenotypes, knowledge about the optimal parameters found to work for a given patient stratum can inform appropriate parameter selection for a new patient falling into that same phenotypic group. Machine learning techniques enable data-driven discovery of phenotypic subgroups. The resulting patient stratification supports improved personalization of neurostimulation treatments compared to a generalized approach applied to the entire heterogeneous population.

In additional example embodiments, the machine learning system is configured to predict therapeutic outcomes, such as the degree of motor symptom improvement, for a wide array of potential stimulation parameter settings rather than directly outputting a single recommended optimal programming. For example, the system estimates the expected clinical outcome for combinations of amplitude values, pulse widths, electrode configurations, a pulse type, a pattern type, a sequence, a duty cycle, and other parameters across the full range of programming options. These predicted outcomes are then utilized to simulate a global search across the multi-dimensional parameter space to identify the settings that maximize therapeutic benefit according to a predefined objective function. This enables flexible optimization of the stimulation parameters for each patient based on their individual symptom profiles and therapeutic goals. By leveraging data-driven outcome predictions across a diverse set of potential settings, the system can determine personalized programming to achieve optimal results without needing to empirically test each parameter combination invasively. The machine learning architecture provides a virtual optimization framework to identify ideal settings tailored to the individual.

The present invention utilizes machine learning algorithms, such as deep neural networks (DNNs), convolutional neural networks (CNNs), recurrent neural networks (RNNs), autoencoders, clustering algorithms, decision trees, random forests, and support vector machines (SVMs), to analyze multimodal imaging data and extract meaningful biomarkers used to determine optimal deep brain stimulation parameters tailored to an individual patient's disease profile and anatomy.

According to example embodiments, systems train one or more learning models with MRI scans and/or other imaging data based on a variety of inputs, such as different targets, different fields of view (FoV), different achieved lead/target relationships, or the like. For example, example systems can train learning model(s) with lead and anatomy data including varying inputs such as anatomical targets, FoV, windows about lead(s), input labels (e.g., electrode types, locations, etc.), and/or features. The systems can provide a variety of output, such as intended target, need for surgical revision, acute outcomes of performance, or the like. In addition, systems can deploy to customer facing software to be integrated with other aggregated data (e.g., BRAINLAB ELEMENTS®).

According to examples of the present disclosure, systems can train one or more networks on imaging and other data to predict optimal stimulation settings in order to predict optimal stimulation parameters on a new patient. For example, MRIs from a region of interest (ROI) around one or more leads with one or more nuclei. Additional example embodiments can use an offline (e.g., simulated) algorithm-guided DBS-programming based on external sensor feedback (e.g., closed-loop programming evaluation using external response (CLOVER (e.g., STIM SEARCH)) to streamline and facilitate programming of DBS using wearable feedback compared to individualized or aggregated programming.

According to examples of training a network to predict best stimulation settings, the network does not need a common space and will learn what features of the imaging data matter. The network does not need segmentations of the patient's brain (although some examples can include such segmentations in addition to or instead of MRI data) and does not need SFMs (although some examples can use them or other E-fields, second derivatives, or the like). In addition, examples of the network can be hierarchical, such as level one providing best contact level, level two providing learning best contact fraction, level three providing learning best amplitude, or the like. Additional examples of the network can include data other than MRI data and other than imaging.

Example embodiments provide advantages over previous techniques by including the ability to assess similarities between patients' imaging data in a native anatomical space, without warping or registering the images to a standard atlas template. By avoiding atlas registration, the examples retain subtle patient-specific anatomical nuances that may be distorted or lost when forcing images into a common coordinate system. Furthermore, bypassing the computationally demanding process of warping all patients' images into atlas space substantially reduces processing time and removes reliance on proprietary atlas registration software tools. Since no predefined atlases are necessarily required (although may be added), example embodiments can incorporate a wider variety of imaging modalities beyond those with existing templates. Additionally, the use of native imaging space enables more seamless integration of non-imaging clinical data, as no alignment to an atlas is necessary; the avoidance of atlas warping simplifies the computational pipeline while preserving critical anatomical details, providing significant advantages over standard atlas-based approaches to image analysis. Furthermore, example embodiments of the present disclosure provide for a next-generation pipeline for incorporating additional datasets as different patients' information is received and can reduce reliance on external aggregation software.

Other and further aspects and features of the invention will be evident from reading the following detailed description of the preferred embodiments, which are intended to illustrate, not limit, the invention.

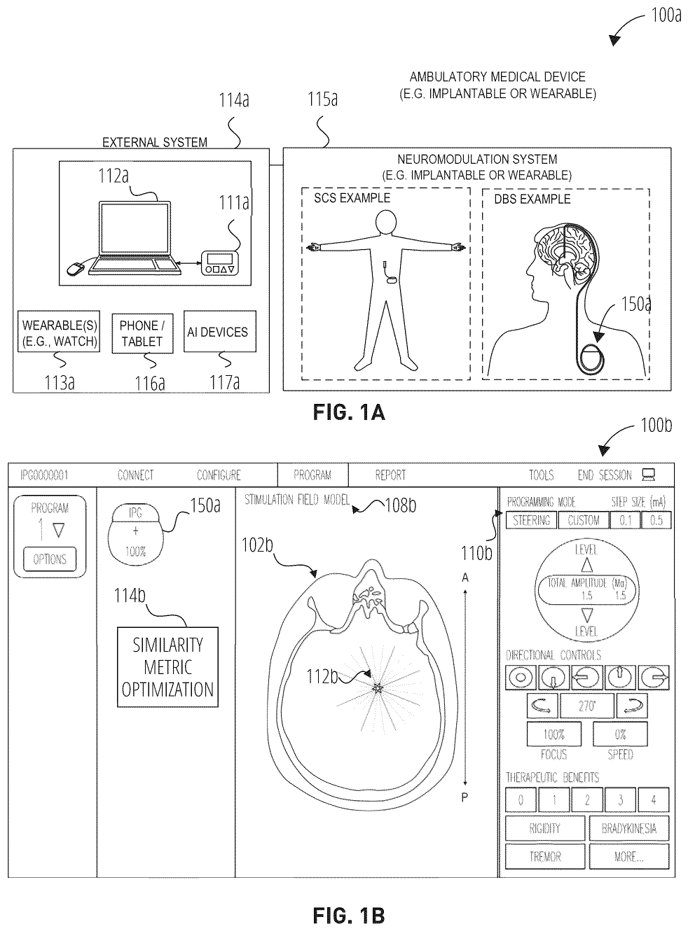

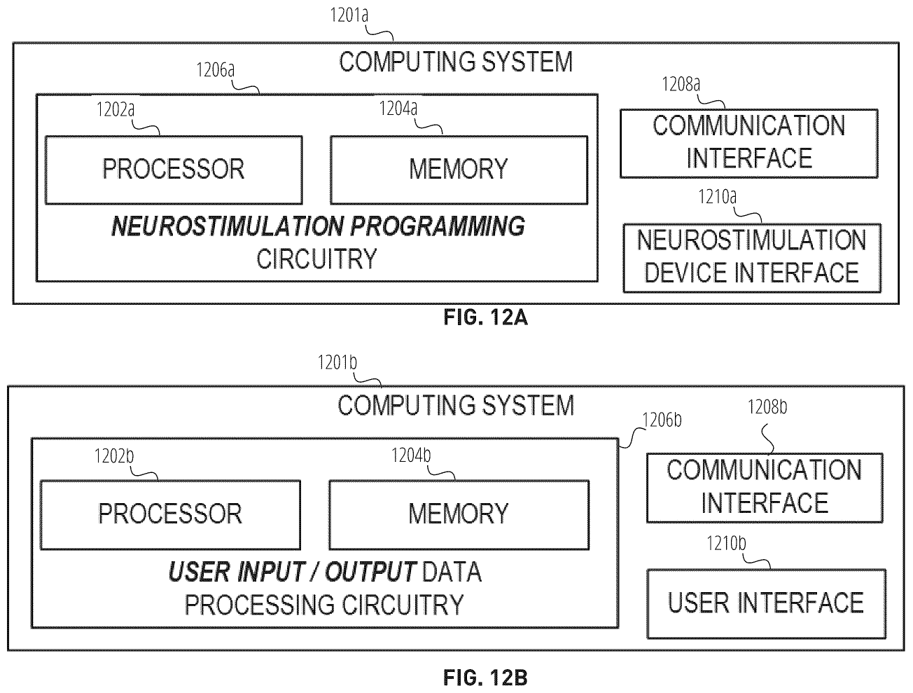

FIG. 1A illustrates, by way of example and not limitation, a block diagram 100a showing a neuromodulation system 115a, for example, like the system depicted and described in connection with FIGS. 12A-B and 13A-B, can be implemented as a spinal cord stimulation (SCS) system or a deep brain stimulation (DBS) system in a patient.

The illustrated neuromodulation system 115a includes an external system 114a that can include at least one programming device. The illustrated external system 114a can include a programmer 111a configured for use by a clinician, patient, device representative, other caregiver, or a combination thereof to communicate with and program the neuromodulator, and a remote control (not shown) configured for use by the patient to communicate with and program the neuromodulator. For example, the remote-control device can allow the patient to turn a therapy on and off and/or can allow the patient to adjust patient-programmable parameter(s) of the plurality of modulation parameters. The external system 114a can further be operatively coupled with one or more patient wearable devices 113a (e.g., watch, ring, brain-sensing monitor, necklace, heartrate monitor, holter monitor, etc.), a patient computing device 116a (e.g., phone, computer, tablet, etc.), and/or patient artificial intelligent (AI) devices 117a (e.g., Amazon® Alexa, Google® Assistant).

FIG. 1A illustrates a medical device as an ambulatory medical device. Examples of ambulatory devices include wearable or implantable neuromodulators. The external system 114a can include a network of computers, including computer(s) remotely located from the ambulatory medical device that are capable of communicating via one or more communication networks with the programmer 111a and/or the remote control. The remotely located computer(s) 112a and the ambulatory medical device can be configured to communicate with each other via another external device such as the programmer 111a or the remote control. The remote-control device and/or the programmer 111a can allow a user (e.g., patient and/or clinician or device rep) to answer questions as part of the data collection process. The ambulatory medical device may include internal or external sensors that can be used to collect data, which can form at least part of the overall pain data, healthcare-related data, or other patient data to be transferred to a data receiving system. Parameter data for the programmed therapy can form at least part of the healthcare-related data to be transferred to a data receiving system.

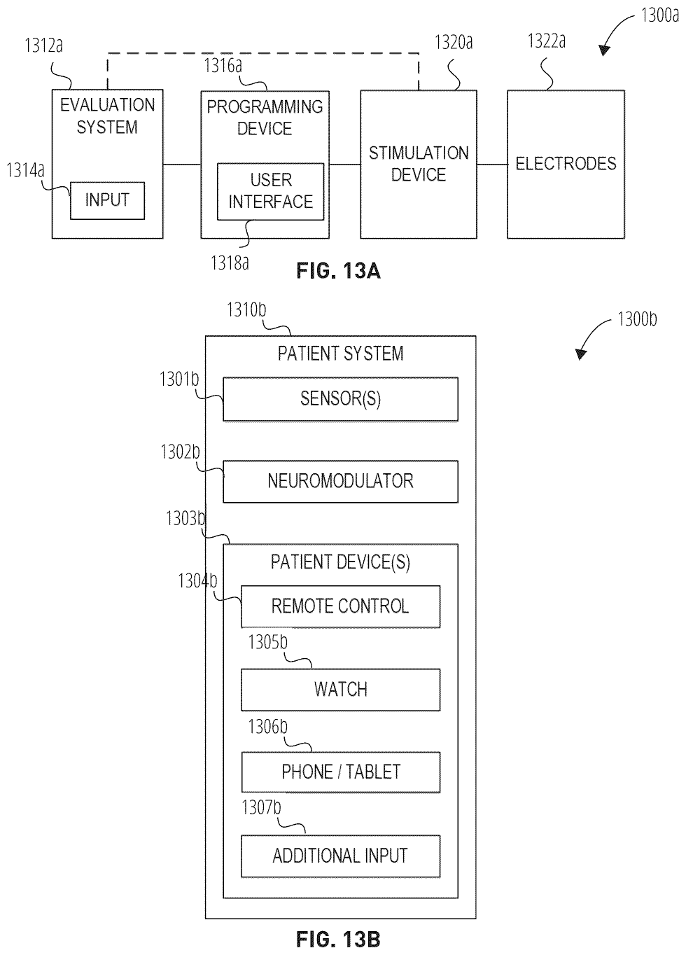

The neuromodulation system 115a, by way of example, can include an implantable device 150a and a lead system. The implantable device 150a may represent an example of the programming device 1316a and/or the stimulation device 1320a as described and depicted in connection with FIG. 13A. The implantable device 150a may include a stimulation output circuit that may produce and/or deliver a neuromodulation waveform. Such waveforms may include different waveform shapes. The waveform shapes may include regular shapes (e.g., square, sinusoidal, triangular, saw tooth, and the like) or irregular shapes. A stimulation control circuit may control which electrodes are used to deliver stimulation and may control the delivery of the neuromodulation waveform using the plurality of stimulation parameters, which specifies a pattern of the neuromodulation waveform. The lead system may include one or more leads each configured to be electrically connected to stimulation device and a plurality of electrodes distributed in the one or more leads. In an example, the number of leads and/or the number of electrodes on each lead depend on, for example, the distribution of target(s) of the neuromodulation and the need for controlling the distribution of electric field at each target.

FIG. 1B includes a user interface 100b for displaying one or more settings associated with an IPG 150a and an implantable lead system, the user interface 100b providing parameter values and/or setting values that can lead to beneficial clinical effects or detrimental clinical effects, in accordance with an example embodiment.

According to the example embodiment of FIG. 1B, a neurostimulation system displayed in the user interface 100b may use a computer-generated three-dimensional (3D) voxelized model to determine a first metric value over a plurality of physiologic structures, analytically derived or user-selected regions (e.g., of the brain or other areas), or combinations thereof. The neurostimulation system may determine one or more stimulation parameters that correspond to the first metric value, such as a stimulation current and an electrical current fractionalization across a plurality of electrodes of the IPG 150a. The first metric value may be referred to as a best metric value that exceeds a threshold metric value, or the largest metric value under a specific electrical current fractionalization. A metric value calculator (not shown) may be configured to determine, for each of the regions, a respective metric value (MV) using the received 3D voxelized model. The MV represents a clinical effect of electrostimulation on the tissue according to a stimulation current and fractionalization of electrical current. In an example, the MV may be computed using a weighted combination of the volumes of the array of 3D voxels in the voxelized model. The current fractionalization refers to current distribution among electrodes, and may be represented by percentage cathodic current, percentage anodic current, or off (no current allocation). Although current fractionalization is discussed in this document, it is to be understood that voltage or electrical energy may similarly be fractionalized among the electrodes, which may result in a particular spatial distribution of the stimulation field.

According to examples, the neurostimulation system displayed in the user interface 100b may determine a stimulation configuration including a stimulation location 112b. The MRI of patient's brain 102b illustrated in the stimulation field model (SFM) 108b may be represented by coordinates in a coordinate space and corresponds to the best metric value. In various examples, the neurostimulation system may determine a virtual electrode state including one or more steering parameters 110b. Through the user interface 100b, a user (e.g., surgeon, practitioner, clinician, etc.) may steer a virtual electrode according to the one or more virtual electrode steering parameters 110b. The system may determine electrical current fractionalization across a plurality of electrodes based on the voltage field of the virtual electrode.

The neurostimulation energy may be delivered according to specified (e.g., programmed) modulation parameters. Examples of setting modulation parameters may include, among other things, selecting the electrodes or electrode combinations used in the stimulation, configuring an electrode or electrodes as the anode or the cathode for the stimulation, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc. The modulation parameters may additionally include fractionalization across electrodes. The fractionalization specifies distribution (e.g., the percentage) of the stimulation current, voltage, or electrical energy provided by an electrode or electrode combination, which affect the spatial distribution of the resultant stimulation field. In an example, current fractionalization specifies percentage cathodic current, percentage anodic current, or off (no current allocation).

In the illustrated example of the user interface 100b, the IPG 150a and the implantable lead system may provide Deep Brain Stimulation (DBS) to a patient at a stimulation location 112b, with the stimulation target being, for example, neuronal tissue in a subdivision of the thalamus of the patient's brain as illustrated in an MRI of a patient's brain 102b. Other examples of DBS targets include, for example and without limitation, neuronal tissue of the globus pallidus (GPi), the subthalamic nucleus (STN), the pedunculopontine nucleus (PPN), substantia nigra pars reticulate (SNr), cortex, globus pallidus externus (GPe), medial forebrain bundle (MFB), periaquaductal gray (PAG), periventricular gray (PVG), habenula, subgenual cingulate, ventral intermediate nucleus (VIM), anterior nucleus (AN), other nuclei of the thalamus, zona incerta, ventral capsule, ventral striatum, nucleus accumbens, white matter tracts connecting these and other structures. The DBS targets may also include regions determined analytically based on side effects or benefits observed in one or more patients and used to identify automatic DBS stimulation settings based on previously gathered and organized data.

Example embodiments of the user interface 100b provide for a similarity metric optimization 114b to assess similarities between patients' imaging data in a native anatomical space, without warping or registering the images to a standard atlas template. By avoiding atlas registration, the examples retain subtle patient-specific anatomical nuances that may be distorted or lost when forcing images into a common coordinate system. Furthermore, bypassing the computationally demanding process of warping all patients' images into atlas space substantially reduces processing time and removes reliance on proprietary atlas registration software tools. Since no predefined atlases are necessarily required, example embodiments can incorporate a wider variety of imaging modalities beyond those with existing templates. The use of native imaging space enables more seamless integration of non-imaging clinical data, as no alignment to an atlas is necessary; the avoidance of atlas warping simplifies the computational pipeline while preserving critical anatomical details.

In device-based neuromodulation therapy, finding an “optimal” stimulation target is an important yet challenging technical problem. It is generally recognized that there is no universal optimal target or desired evoked response due to differences in patient condition or disorder, anatomy (anatomical target), lead and trajectory, center (imaging equipment position, etc.), surgeon (how they implant and position lead, their preferences for lead placement), or symptoms to improve (e.g., tremor to cognitive skill improvement). In DBS for Parkinson's Disease (PD) management, there is no consensus on the “best” DBS target across PD patients. While DBS effectively treats several motor symptoms of PD, limitations remain in side effect profile, management of non-motor symptom, accurate lead placement with intraoperative testing, and selection of optimal stimulation parameters. DBS may improve motor symptoms in some patients with advanced Parkinson's Disease (PD), and other motor and non-motor disorders. Stimulation leads/electrodes for PD treatment are commonly implanted in subthalamic nucleus (STN) or globus pallidus internus (GPi), although other neural targets, such as pedunculopontine nucleus (PPN) and posterior subthalamic area (PSA), and others, have been shown as effective targets for Parkinsonian tremor control and control of other Parkinson's symptoms in patients. However, optimal target(s) of DBS to manage PD and other movement and non-movement disorders may vary across patients, and example embodiments of the user interface 100b may help identify a “universal” or “optimal” DBS target. A DBS system capable of providing DBS setting optimization to calculate similarity metrics between patients with similar conditions, anatomy, pain levels, or the like is provided by a similarity metric optimization 114b.

According to examples of the present disclosure, one or more machine learning algorithms may be used to improve and/or automate DBS programming by using previously gathered and organized data, including data from a plurality of patients, based on, for example, a plurality of assessments (e.g., local field potential measurements from the implanted leads, anatomical placement of the leads based on MRI and CT images, motor diary information, unified Parkinson's disease rating scale (UPDRS) scores, quantitative assessments using wearable accelerometers, speech recordings, timed motor tests), at a plurality of neurostimulation settings, and then using the stored data to calculate one or more similarity metrics between patients and use these similarity metrics, individually or in combination, to determine stimulation parameter values with a higher likelihood of beneficial clinical effect and/or lower likelihood of detrimental clinical effect(s). The calculated similarity metrics, as calculated by the similarity metric optimization 114b, can be used to recommend a setting based on the entirety of these data, rather than making serial adjustments after one or two observations.

Similar approaches described herein may be used for deep brain stimulation, and other conditions, for example movement disorders such as dystonia, which is further complicated by the slow onset of DBS response and resulting difficulty of adjusting using serial observations; or essential tremor, which may show optimal tremor control for different parts of the body at slightly different stimulation settings. Cognitive disorders include ailments such as Alzheimer's disease or Parkinson's-related dementia. DBS is also used to affect structures, such as the fornix, nucleus basalis Meynert, or entorhinal cortex. Cognitive performance is complex and may be assessed through a wide variety of methods, including working memory tasks (e.g., N-back tests, mini-cog), questionnaires and rating scales (mini-mental state examination (MMSE), Mattis Dementia Rating Scale, Alzheimer's Disease Assessment Scale-Cognition (ADAS-cog), etc.), brain imaging, mood assessments, and dual motor-cognitive tasks. Furthermore, like movement disorder evaluations, cognitive performance may be time-consuming to assess and does not lend itself to programming through serial observations.

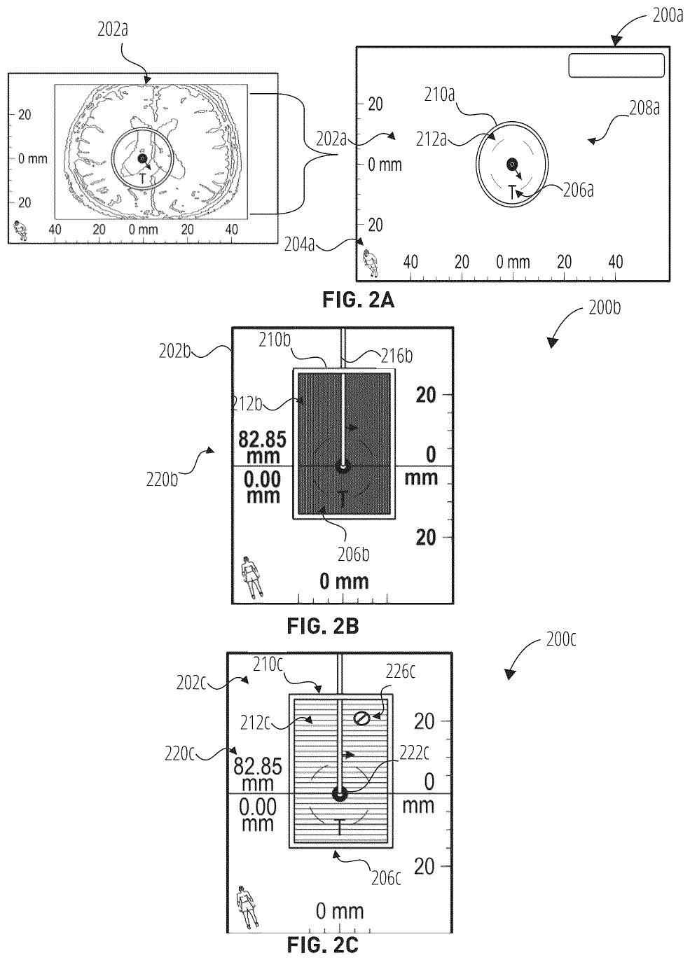

FIGS. 2A-2C illustrate, by way of example, a series of radiographic imaging images identifying regions of interest in a patient to determine stimulation setting parameters, in accordance with one embodiment. The examples of FIGS. 2A-2C illustrate, by way of example and not limitation, similarities based on gray intensity of MRI and CT imaging to identify similarity metric(s). According to these examples of identifying similarity metrics, no automatic brain object segmentation software is needed. For these similarity metrics, a region of interest (ROI) is defined (e.g., a cylinder around a lead represented by circles and rectangles in FIGS. 2A-2C), where the similarity metric is based on the similarity in the gray intensity of voxels within the ROI of different patients.

According to examples, similarity metrics can be based on, for example and not limitation, a database of medical imaging data from the plurality of patients corresponding to therapeutic outcomes based on previously applied neurostimulation parameters, and wherein the one or more similarity metrics are calculated directly from the multimodal medical imaging data without requiring spatial normalization or warping to a common coordinate system. For example, a target simulation parameter can be used to find or identify one or more optimal, acceptable sufficient, preferred, therapeutic, or the like parameter used according to examples of the present disclosure. In some examples, the target stimulation parameter(s) can be sub-optimal settings to guide in avoidance of certain metrics or outcomes. According to additional examples, the system may use more than one setting or parameter with equivalent or near-equivalent (e.g., goodness) stimulation parameters. For example, in some patients, a best level of achievement may be less than desired.

Examples of the present disclosure as shown in FIGS. 2A-2C provide for deep learning approaches to be utilized in targeting to provide patient-specific segmentation of various nuclei relevant to neuromodulation systems, such as nuclei relevant to DBS. The systems proposed throughout directly utilize information available from voxel intensities from imaging data in areas immediately adjacent to one or more implanted lead(s) directly from the patient's native space imaging without any normalization to a standard space or atlas space. For exemplary purposes in this description, a similarity metric based on a patient's symptomatology and response to therapy can be based on distance metrics between the patient's symptom scores and/or symptom response to therapy. One or more similarity metrics can be used according to different embodiments. Although it will be understood by one of ordinary skill in the art that other metrics can similarly be applied; for example, the similarity metric can be changed to other metrics, such as, for example, an anatomy-based similarity metric, a patient's symptomatology similarity metric, a patient's response to therapy similarity metric, or the like.

Example embodiments of the present disclosure include the ability to incorporate a diverse range of imaging modalities when calculating similarity metrics between patients in native anatomical space. Structural imaging methods such as MRI (e.g., T1, T2, proton density, etc.) and CT provide high-resolution anatomical data. Functional modalities such as functional MRI and PET offer comparisons of metabolic or disease-specific patterns. Diffusion MRI maps connectivity via white matter tracts. Other applicable modalities include SPECT, ultrasound, optical imaging, and multimodal combinations. No predefined atlases tailored to specific modalities are required, granting substantial flexibility. Both structural and functional imaging types containing raw pixel data can be analyzed to derive similarity metrics between corresponding patient images. This enables a rich characterization of anatomical and physiological commonalities and differences among patient cohorts from a diverse array of imaging perspectives.

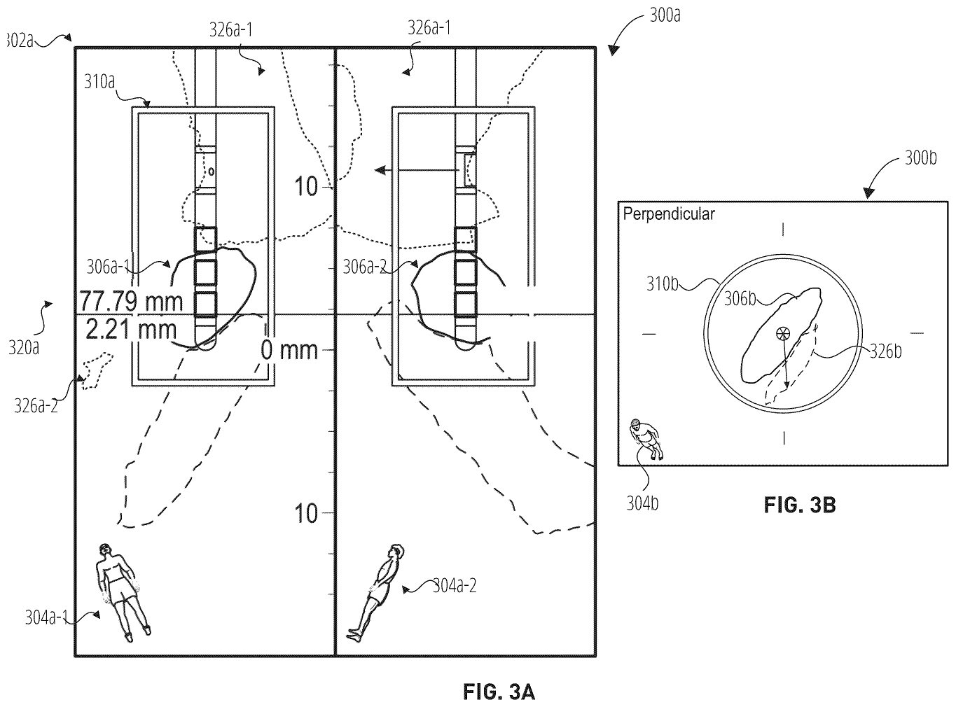

Turning to FIG. 2A is a block diagram 200a, by way of example and not limitation, identifying a region of interest (ROI) 210a in a gray area 208a of an MRI image 202a, in accordance with example embodiments of the present disclosure. As shown in block diagram 200a, MRI image 202a includes a radiographic scan of a patient's brain; however, for simplicity of explanation in FIGS. 2A-2C and 3A-3B, the image of the brain is removed from the figures.

Since no alignment to a common atlas space is required, any relevant clinical, demographic, genetic, physiological, or behavioral data can be integrated with the native imaging data to enhance similarity determinations. Example embodiments include calculating similarity metrics directly from native imaging space, such as MRI image 202a, including the ability to seamlessly integrate non-imaging data modalities. These include other metrics or data, such as clinical assessments (e.g., symptom severity scores, cognitive evaluations, motor function tests); medication history (dosages, timing, types); genetic and electrophysiological data; neurochemical concentrations measured through microdialysis; behavioral data captured via wearable sensors; patient-reported outcomes surveys; disease characteristics (e.g., subtype, stage, comorbidities); and treatment history. Since no alignment to a common coordinate space is necessary, any relevant clinical, demographic, genetic, physiological, or behavioral data can be combined with the native imaging data found in MRI image 202a to enhance multidimensional characterization of similarities and differences among patient cohorts. Example techniques thereby enable a holistic data fusion approach for patient matching that encompasses both anatomical imaging perspectives, according to a variety of orientations such as orientation 204a, and diverse non-imaging data domains.

In the example embodiment of block diagram 200a, the method includes positioning the neurostimulation lead within the patient relative to a target tissue region, such as target 206a (represented in FIGS. 2A-2C with a “T”). The target tissue region may be a spinal cord, a posterior longitudinal ligament, white matter, or gray matter. For example, positioning the neurostimulation lead within the patient may include positioning the neurostimulation lead in a gray area 208a that includes a gray intensity 212a such that the target tissue region is the brain. In the example of block diagram 200a, positioning the neurostimulation lead within the patient may include positioning the neurostimulation lead in a brain, such that the target tissue region is gray matter or white matter with the target tissue region being target 206a. In still another example, positioning the neurostimulation lead within the patient may include positioning the neurostimulation lead in a brain, such that the target tissue region is gray matter or white matter. In still another embodiment, the neurostimulation lead may be implanted within a brain of the patient with one of the electrodes adjacent to white matter and the other electrode adjacent to gray matter, and the target tissue region may be the white matter or the gray matter. In another example, positioning the neurostimulation lead within the patient may include positioning the neurostimulation lead in a ventral region of the epidural space such that the target tissue region is the posterior longitudinal ligament. In yet another example, positioning the neurostimulation lead within the patient may include positioning the neurostimulation lead between the spinal cord and a dura, such that the target tissue region is the spinal cord.

FIG. 2B is a block diagram 200b, by way of example and not limitation, identifying a region of interest (ROI) 210b in a heavy (e.g., dark) gray area 216b of an MRI image 202b, in accordance with example embodiments of the present disclosure.

Magnetic resonance imaging (MRI) produces voxel-level quantitative maps of inherent tissue gray intensity, such as gray intensity 212b, providing a critical feature for computational analysis and automated processing. Specifically, MRI gray intensity denotes the brightness of each volumetric pixel (voxel) on a quantitative scale ranging from black to white, dependent on tissue-specific parameters including proton density, T1 and T2 relaxation times. This allows differentiation of anatomical structures based on their characteristic intensity signatures. However, MRI gray values are also influenced by user-defined scanning variables such as echo time (TE) and repetition time (TR), requiring normalization to account for inter-patient variations. While useful for tissue characterization, MRI intensity values are prone to artifacts and noise which affect intensity uniformity. Nonetheless, local variations in MRI gray intensity within a region of interest, such as ROI 210b, can indicate pathology or lesions. Through examination of MRI voxel-level gray values, the disclosed technique exploits tissue-specific intensity profiles for tasks including automated segmentation, registration, and anatomical pattern recognition, thereby deriving clinically relevant information from this fundamental image feature.

A key element of the disclosed technique is the analysis of voxel intensities within medical imaging data, such as MRI or CT scans. Voxels are the three-dimensional equivalent of pixels, representing small volumetric units within the imaging data. The intensity of each voxel refers to its numeric brightness value, which corresponds to quantitative properties of the underlying tissue in that location. For instance, MRI voxel intensities reflect proton density and relaxation times, while CT voxel intensities indicate radiodensity and X-ray attenuation. Thus, voxel intensities provide localized quantitative information about tissue characteristics. Higher intensity values typically correspond to brighter voxels in the scan image, while lower intensities are darker voxels. However, the mapping between intensity values and visible brightness is scanner specific. Voxel intensities act as crucial input data for radiomic analysis approaches which extract texture and histogram-based features. Example embodiments of the disclosed technique leverages voxel intensity patterns within medical imaging data, such as MRI or CT scans to assess tissue-level similarities between patient cohorts for optimized therapy predictions.

Voxels represent fundamental three-dimensional volumetric units used in medical imaging and other volumetric data representations. Specifically, a voxel defines a small regular volumetric grid element in three-dimensional space, analogous to a two-dimensional pixel. In medical imaging, each voxel encodes the image intensity or density value of a corresponding minuscule cubic volume, such as a 1 mm×1 mm×1 mm cube, within the overall three-dimensional scan volume. In the example block diagram 200b, the cubic volume value 220b identifies 82.85 mm as the ROI 210b of MRI image 202b. The size of voxels determines the resolution of three-dimensional imaging data-smaller voxel sizes enable higher resolution. Advanced analysis and visualization techniques for medical scan data, including segmentation, registration, and morphometry, rely on the voxel structure to delineate anatomical boundaries in three dimensions and quantify local tissue concentrations.

A key aspect of the disclosed technique is the use of voxel-level intensity data to quantify and assess anatomical targets identified in medical imaging such as MRI scans. Intensity characteristics within and surrounding the target region, such as target region 206b, provide information about tissue composition and morphology. For instance, average target intensity, intensity homogeneity, gradients along boundaries, and bilateral comparisons reveal properties of the underlying anatomy. Multi-spectral intensity analysis across different MRI sequences provides additional tissue information (not shown). For example, texture filters can be applied to intensity patterns to discern structural complexity. The rich information encoded in voxel intensities enables in-depth analysis and sensitive characterization of anatomical targets beyond simple segmentation. This intensity-based approach facilitates precision targeting and therapy optimization without requiring whole-brain atlas registration.

FIG. 2C is a block diagram 200c, by way of example and not limitation, identifying a region of interest (ROI) 210c in a less heavy (e.g., light) gray area 212c of an MRI image 202c, in accordance with example embodiments of the present disclosure.

In one embodiment, the neurostimulation system utilizes a computer-generated three-dimensional (3D) voxelized model of MRI image 202c to calculate a first metric value 220c across multiple anatomical structures, regions, or combinations thereof. The system then determines corresponding stimulation parameters, including, for example, stimulation current and fractionalization of electrical current across multiple electrodes, which yield the first metric value 220c. This first metric value 220c may constitute the maximum achievable value given a particular current fractionalization. Current fractionalization refers to the distribution of current among the electrodes, represented as percentages of cathodic and anodic current per electrode or an off state with no current. Analogously, voltage or electrical energy can be fractionalized among electrodes to produce a desired spatial distribution of the stimulation field.

Additionally, the system can determine an optimal stimulation location 222c represented by coordinates in a coordinate space that corresponds to the best, or maximal, first metric value 220c. Additional examples of the system may also determine virtual electrode steering parameters and enable user steering of a virtual electrode via an interface. This in turn allows calculating the electrode current fractionalization based on the voltage field of the virtual electrode.

The received 3D voxelized anatomical model comprises a plurality of volumetric pixels (voxels) each having a geometric voxel volume and an associated voxel value. The voxel volume represents the physical size of the voxel. The voxel value indicates properties such as the likelihood that the voxel contributes to a predicted clinical outcome, which may be therapeutic benefit or side effects. For instance, a voxel value of 0.9 signifies an 90% probability that the voxel belongs to a structure tied to a specific outcome. Alternatively, multiple anatomical regions may be represented within the same voxel space, where voxel values denote the relative weighting of each region. This allows differentiating structures like seizure foci versus speech areas. The voxels may be categorized into target regions expected to produce therapeutic effects, and avoidance regions likely to cause side effects. In one embodiment, target region(s), such as target region 206c, and avoidance region(s), such as avoidance region 226c, are encoded in the same voxel space, where voxels in avoidance regions are assigned negative values, while target voxels have positive values. The voxel values thereby delineate boundaries and memberships of anatomical structures which influence clinical outcomes.

A receiver circuit (not shown) may receive a 3D voxelized model of a tissue as a target of electrostimulation, such as particular brain tissue as a target of deep brain stimulation (DBS). A voxel represents a volumetric element of a computerized physiologic structure or analytically determined structure, such as a computerized tissue representation, in a 3D space. A voxel may have specified size in each dimension, such as 0.5 mm or less. The 3D voxelized model may include a computer-generated graphic model representing volumetric tissue elements and their responses to the electrostimulation. In an example, the 3D voxelized model comprises an array of 3D voxels each specified as belonging to one of a plurality of physiologic structures, such as a target region or an avoidance region. The target region 206c may refer to a physiologic structure, analytically derived or user selected regions (e.g., of the brain or other areas), or combinations thereof. The target region 206c may be associated with known therapeutic benefits of the electrostimulation. The avoidance region 226c may refer to a physiologic structure, analytically derived or user selected regions, or combinations thereof that are associated with a known side effect of the electrostimulation.

FIGS. 3A-3B illustrate, by way of example and not limitation, two example radiographic imaging images identifying voxels corresponding to a brain image around a lead, in accordance with one embodiment. The examples of FIGS. 3A-3B illustrate similarities based on gray intensity of MRI and CT imaging to identify similarity metric(s) as described and depicted in connection with FIGS. 2A-2C.

FIG. 3A includes a display screen 300a illustrating a same brain location of an MRI image 302a based on two orientations 304a-1/304a-2 scans, in accordance with example embodiments of the present disclosure.

According to these examples of identifying similarity metrics, each voxel in the ROI 310a/310b is assigned a value based on a brain object (e.g., subthalamic nucleus, substantia nigra, etc.) that it belongs to. The similarity metric is based on the similarity of the voxels within the ROI of different patients. Since no alignment to a common atlas space is required, any relevant clinical, demographic, genetic, physiological, or behavioral data can be integrated with the native imaging data to enhance similarity determinations. Example embodiments include calculating similarity metrics directly from native imaging space, such as 300b, is the ability to seamlessly integrate non-imaging data modalities.

Example embodiments of the present disclosure include the ability to incorporate a diverse range of imaging modalities when calculating similarity metrics between patients in native anatomical space. Structural imaging methods such as MRI (e.g., T1, T2, proton density, etc.) and CT provide high-resolution anatomical data. Functional modalities such as functional MRI and PET offer comparisons of metabolic or disease-specific patterns. Diffusion MRI maps connectivity via white matter tracts. Other applicable modalities include SPECT, ultrasound, optical imaging, and multimodal combinations. According to example embodiments of FIGS. 3A-3B, no predefined atlases tailored to specific modalities are required, granting substantial flexibility. Both structural and functional imaging types containing raw pixel data can be analyzed to derive similarity metrics between corresponding patient images. This enables a rich characterization of anatomical and physiological commonalities and differences among patient cohorts from a diverse array of imaging perspectives.

A key aspect of the disclosed technique is the use of voxel intensity patterns from medical imaging data to quantify anatomical and physiological similarities between patients. This is accomplished by extracting voxel intensities within defined regions of interest (ROIs), such as ROI 310a, for each patient, such as the area surrounding implanted DBS leads. Calculated similarity metrics can include average ROI intensity, intensity histogram correlation, intensity variance, textural feature analysis, clustering algorithms, deep neural network models, and multivariate distance metrics. Patients with more correlated intensity histograms, closer average intensity values, shared cluster membership, smaller multivariate distances, and other similar quantitative metrics are considered to have higher anatomical and physiological similarity. The degree of voxel intensity pattern similarity thereby enables customized groupings of patients for optimized selection of therapeutic parameters tailored to an individual. This data-driven approach leverages voxel-level imaging information to precisely characterize patient cohorts without the need for broader atlas registration.

In additional example embodiments, a localizer box (not shown) can be placed on the frame to generate a coordinate system within which each spot, which can be described using coordinates for laterality (x-axis) anterior-posterior position (y-axis) and superior-inferior position (z-axis). These coordinates are calculated in reference to a line connecting the anterior and posterior border of the displayed anatomical location.

FIG. 3B is a display screen 300b illustrating a same brain location of an MRI image 302a based on orientation 304b of an MRI scan, in accordance with example embodiments of the present disclosure.

The display screen 300b is configured to display one or more of the computer-generated 3D voxelized tissue model, stimulation configuration including various stimulation parameters, or virtual electrodes, among other control parameters. The display screen 300b illustrates a perpendicular orientation 304b of the ROI 310b including a target region 306b and an avoidance region 326b.

In additional examples, clinical effect information associated with stimulation of a particular tissue site may be displayed on the displayed screen, including information about therapeutic benefits and side effects produced by the stimulation. The benefits or side effects may take a form of a score or a graphical representation.

Additional example embodiments include the ability to fuse multimodal data for comprehensive similarity analysis, as no common coordinate space is required. For example, structural MRI scans depicting anatomy could be combined with diffusion MRI tractography showing white matter connectivity patterns. Areas of overlapping fiber tracts between patients could represent an additional similarity metric. Functional MRI maps of brain activity during motor tasks could be integrated with behavioral motion sensor data, comparing similarities in activation locations and movement patterns. PET imaging of dopamine transporter availability could be correlated with genetic polymorphisms affecting dopamine regulation, pairing metabolic and genetic biomarkers. CT scans could be augmented with clinical symptom severity scores, comparing anatomical patterns with phenotypic disease profiles. The fusion of diverse data types into unified characterizations of patient physiology enables robust similarity determinations that leverage relationships between genetic, molecular, anatomical, and behavioral domains. Rather than relying on imaging alone, example techniques allow for holistic integration of multimodal data for precision patient clustering and personalized therapy predictions.

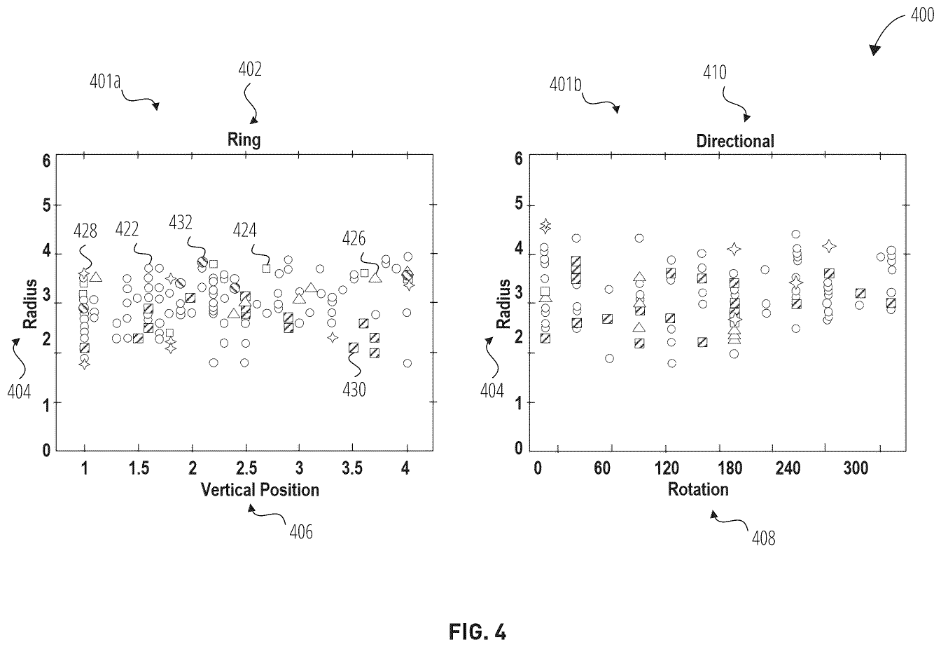

FIG. 4 is a block diagram 400 illustrating two panels showing correlations between patients based on stimulation fields, in accordance with example embodiments of the present disclosure.

A first panel 401a is a possible stimulation field based on vertical positions and radii for ring mode stimulation settings 402. In the first panel 401a, the x-axis represents vertical positions 406 and the y-axis represents radii 404. In each panel, a variety of shapes (e.g., stars, triangles, squares, etc.) represent patients being similar to each other, and circles 422 represent all of the remaining patients. For example, in the first panel 401a and the second panel 401b, squares 424 represent a first subset of patients that includes patients with a first similarity metric in common, tringles 426 represent a second subset of patients that includes patients with a second similarity metric in common, stars 428 represent a third subset of patients that includes patients with a third similarity metric in common, patterned squares 430 represent a fourth subset of patients that includes patients with a fourth similarity metric in common, and patterned circles 432 represent a fifth subset of patients that includes patients with a fifth similarity metric in common. It will be understood by those of skill in the art that each subset of patients can include one or more similarity metrics in common, including, for example, overlapping similarity metric(s), related metrics, similar beneficial clinical effects, similar detrimental clinical effects, or other commonalities or dissimilarities.

In one embodiment, the system utilizes dissimilarity or distance metrics to measure differences between patients' brain anatomy, disease characteristics, outcomes, and/or other dissimilar metrics to identify optimal stimulation parameters. These complement similarity metrics used to identify commonalities between patients. Exemplary distance metrics include, for example and not limitation, imaging distance to quantify anatomical variations based on MRI/CT scans; disease profile distance to determine dissimilarity of symptoms, progression, and comorbidities; outcome distance to compare differences in symptom severity, side effects, and quality of life measures; and parameter distance to directly contrast the stimulation voltage, pulse width, and frequency settings. Patients with remarkably high dissimilarity, indicating substantial anatomical, disease, outcome, or optimal parameter differences, may be excluded from patient clusters used to guide programming for a new patient. The distance metrics thereby filter out highly dissimilar patients prior to similarity analysis to improve stimulation recommendations. For example, the circles 422 can include patients with dissimilar metrics that can be excluded from the similarity metric calculations. In alternative example embodiments, one or more dissimiliar metrics may be included in a portion of metric calculations.

The block diagram 400 illustrates two panels showing subsets of patient groupings based on similarity analysis for determining optimal stimulation parameters. The disclosed technique utilizes two primary stimulation modalities to optimize the spatial targeting of deep brain structures. The different subsets signify patients with one or more similarity metric(s), while circles 422 denote all other patients in the overall dataset.

For example, the left panel depicts a potential stimulation field based on vertical positions and radii using a ring mode stimulation approach. For example, the ring mode stimulation approach 402 can utilize multiple electrode contacts concurrently to control dorso-ventral location and radius of the stimulated tissue volume. In the ring mode stimulation technique 402, multiple electrode contacts are activated concurrently along the lead to control the dorso-ventral center and radius of the stimulated tissue volume. Specifically, the vertical position 406 of the stimulation field is determined by the selected contacts and their relative weighting, with more ventral contacts pulling the field downward and dorsal contacts shifting it upward. The Radius 404 of the ring-shaped stimulation field 402 is modulated by a total delivered current, with higher amperages producing larger radii.

The second panel 401b is a possible stimulation field based on rotations and radii for directional mode stimulation settings 410. In the second panel 401b, the x-axis represents rotational positions 408 and the y-axis represents radii 404. The right panel shows a possible stimulation field based on rotation angles and radii using a directional stimulation technique 410. Rotational stimulation involves asymmetric weighting of electrodes around the lead to steer the field in medial-lateral and anterior-posterior directions, also controlling radius 404. Directional (or rotational) stimulation 410 involves applying asymmetric weights to electrodes around the lead circumference to steer the field mediolaterally and anteroposteriorly. The rotation angle of the stimulation field depends on the relative contact weighting distribution, with greater current applied to contacts in the desired steering direction. Similar to ring mode, the radius of the rotationally targeted stimulation field is dictated by the total stimulation current. Thus, the subset of patient clusters represents subgroups with common anatomical traits used to derive preferred programming settings, while the circles 422 are dissimilar patients not utilized for parameter selection. This enables customized stimulation protocols tailored to phenotypic patterns.

Either modality thereby allows precise targeting of anatomical structures in three dimensions by selecting appropriate vertical positions 406 and ring radii 404, or rotation angles 408 and radii 404. This field shaping permits customized stimulation fields optimized for patient-specific neuroanatomy.

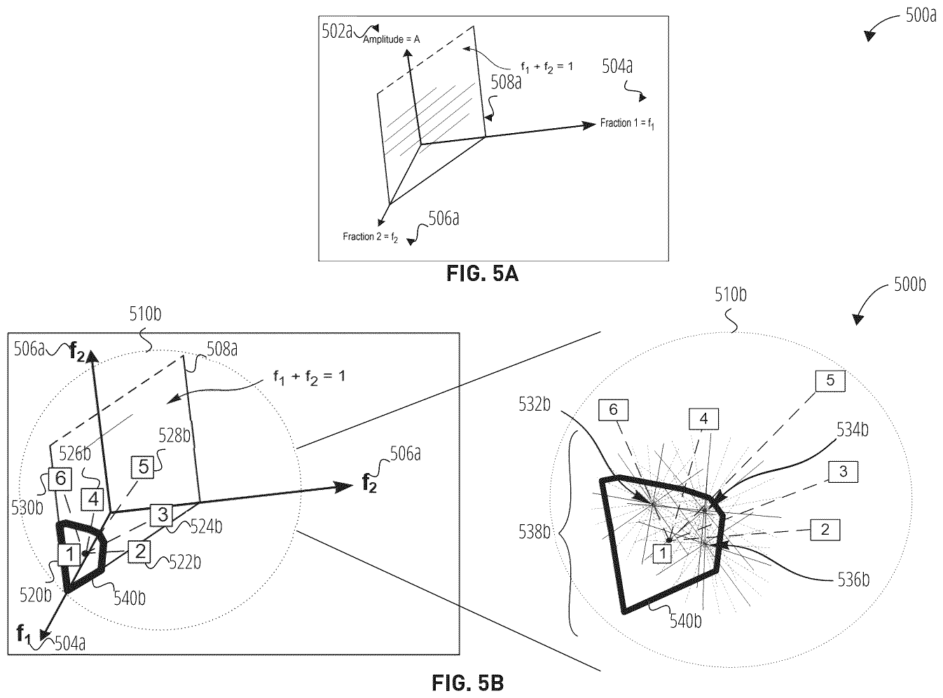

FIGS. 5A-5B illustrate, by way of example and not limitation, block diagrams 500a and 500b depicting empirical determinations of amplitude and fractionalization values used to constrain a search space for a particular patient of interest, in accordance with example embodiments.

FIG. 5A illustrates an example visualization of DBS programming guided by phenotypic similarity, in accordance with one embodiment.

For example, DBS programming can involve determining an optimal or adequate value(s) of the rate, pulse width, amplitude, contact fractionalization, and/or other therapeutic parameters. In some DBS embodiments, the rate and pulse width can be fixed, and the amplitude and fractionalizations can be determined empirically or otherwise. According to some example embodiments, a method to utilize data from other patients can be used to constrain the search space for a particular patient of interest. Such embodiments of FIGS. 5A-5B provide guides for determination of amplitude and fractionalization, which both apply generally to any parameter searched.

In FIG. 5A, for example, suppose rate and pulse width are fixed and a user (e.g., surgeon, technician, etc.) only wants to determine amplitude and fractionalization. For purposes of case in visualization, suppose to that there are only two electrodes (although any number of electrodes or paddles could be used). Based on these suppositions for this example embodiment, the search for best (e.g., ideal, therapeutic, etc.) parameters is a search in a 3-dimensional (3D) space as shown in FIG. 5A, delineating amplitude (A) 502a represented along the y-axis, fraction 1 (f₁) 504a represented along the x-axis, and fraction 2 (f₂) 506a represented along the z-axis. As shown in FIG. 5A, the search is a search on a plane created in this 3D space, where the search is on plane 508a. In examples including more than two electrodes, the plane becomes a hyperplane in a higher dimensional space.

In one embodiment, the system builds computational models from patient-specific anatomical imaging data to simulate the effects of different stimulation parameter settings and calculate corresponding metric values. These models enable computationally testing a wide range of parameters to determine optimal combinations that maximize a desired metric. For instance, the volume of tissue activated (VTA) for a given parameter set can be calculated as one metric value, where higher overlap with therapeutic targets indicates more effective stimulation. Machine learning techniques may also be employed to analyze relationships between patient data, parameter settings, and outcomes in a training dataset, in order to predict optimal parameters for new patients. The metrics computed may include factors such as volume of activation overlap with anatomical targets, avoidance of side effect regions, minimization of stimulation current or energy, and/or other measures of therapeutic potency and efficiency. The computational modeling thereby allows high-throughput evaluation of parameter settings to determine those yielding the most favorable metric values for activating targets while avoiding side effects.

It will be understood by those having skill in the art that DBS programming guided by phenotypic similarity, and/or other algorithms and models according to examples presented herein can alternatively, additionally, or otherwise employ open-loop machine learning for implementing parameterization for sub-perception therapy according to examples of the present disclosure. However, for simplicity in explanation, closed-loop algorithms are used without limitation. Open loop machine learning refers to models that are trained on a fixed dataset and deployed without any feedback loop. In open loop systems, models make predictions on new input data but do not use those predictions to further update themselves. The learning is open without any feedback flow. Open loop models are trained on historical training data, validated, and then deployed statically. They do not change or adapt once deployed.

FIG. 5B is a block diagram 500b illustrating an example embodiment of the plane 508a in a 3-dimensional (3D) space delineating fraction 2 (f₂) 506a represented along the y-axis, fraction 2 (f₂) 506a represented along the x-axis, and fraction 1 (f₁) 504a represented along the z-axis. including additional patients' data, in accordance with one embodiment.

In the block diagram 500b, suppose the system has data from additional patients about their best (e.g., ideal therapeutic values) fractionalization and amplitudes. In such an example, a basic prescription on how to use this data to constrain a parameter search in a new patient is provided. For example, the prescription is to compute the similarity between the new patient's data and the data from the existing patients and/or artificial patient data (e.g., simulated patient data). This similarity can be computed using any of the existing data from the new and/or previously programmed patients.

For example, the similarity can be computed using the imaging data alone, or it could be computed by incorporating other parameters, such as, for example and not limitation, types of symptoms, Levodopa responsiveness, etc. The outcome being one or more similarity metric(s) that rank the existing datasets, or a subset of the existing datasets, by how similar they are to the new patient's data. Given this ranking by similarity, the system according to FIGS. 5A and 5B can now constrain the search space of stimulation settings for the new patient.

For example, the plane 508a is created by the expression f₁+f₂=1 from FIG. 5A, including additional patient data, such as similarity point 1 520b, similarity point 2 522b, similarity point 3 524b, similarity point 4 526b, similarity point 5 528b, and similarity point 6 530b. The similarity points and resulting region 540b are displayed in block diagram 500b in the enlarged area 510b. Assuming, without loss of generality, that the similarity points in FIG. 5B are labeled from most to least similar (e.g., 1 is most similar and 6 is least similar), the system can propose the following algorithmic calculations for determining ideal therapeutic setting parameters:

First step: Connect point 1 520b and point 2 522b with a line and draw a line perpendicular to this line that bisects the search space into multiple regions, such as a first region 532b that is closer to similarity point 1 520b and a second region 536b that is closer to similarity point 2 522b. For exemplary purposes, each of the perpendicular lines bisecting the search space are illustrated in the enlarged area 510b as dotted lines, identified altogether as perpendicular lines 538b.

Second step: Repeat the first step for lines joining similarity point 1 520b to similarity point 3 524b, similarity point 1 520b to similarity point 4 526b, etcetera for as many points of similarity data exist. For example, a third region 534b is shown identifying points closer to similarity point 5 528b.

Third step: Each step produces (e.g., provides the system and/or user) with a sub-region of the search space and the intersection of these sub-regions gives a final constrained search space. For example, in the example of FIG. 5B, the final constrained search space results in the region 540b circumscribed by the solid line.

Fourth step: The more data that is provided from other patients (e.g., similarity points 1-6) and/or artificially generated patient data, the more constrained the search becomes and the tighter the constraints.

Fifth step: Instead of using bisectors, the system according to FIGS. 5A and 5B can also identify or draw the separations at a location proportional to the similarities.

It will be understood by those having ordinary skill in the art that steps 1-5 above may be combined in one or more combinations according to additional example embodiments, excluding one or more steps.

For example, empirical determinations of amplitude and fractionalization values used to constrain a search space for a particular patient of interest can include a showing of high amplitude, low amplitude, less fractionalization, and more fractionalization. A patient of interest can have a patient amplitude range from [1 mA, 4 mA] and a patient fractionalization range from [0%, 50%]. A search space along the x-axis can include fractionalization [0%, 50%] and along the y-axis can include amplitude [1 mA, 4 mA]. A shaded region 540b shows the constrained search space for the patient of interest based on empirically determined ranges of effective and safe amplitude and fractionalization values. This reduces the parameters that need to be tested for this individual patient of interest according to patient similarity data. Such an example shows empirical amplitude and fractionalization values for a sample patient of interest used to define ranges for these parameters. This constrains the search space, bounded by the determined amplitude and fractionalization limits, which contains candidate solutions likely to be effective and safe for this patient of interest.

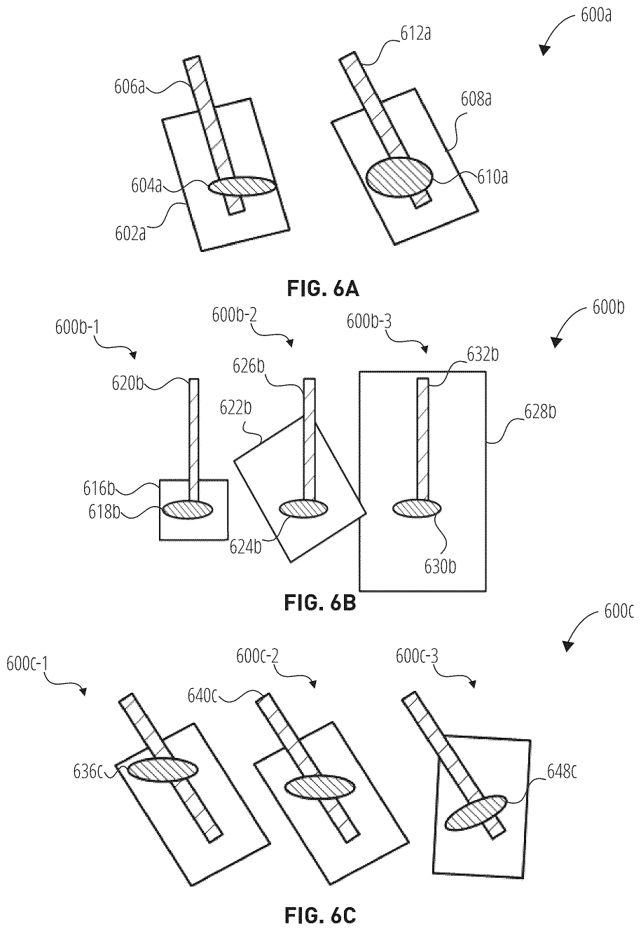

FIGS. 6A-6C illustrate example embodiments of a variety of targets to exemplify how to train learning models using imaging data according to a variety of inputs and/or in order to receive a variety of outputs, in accordance with some embodiments. According to examples of the present disclosure, a system employing one or more learning models, such as a deep neural network, can be used to predict stimulation parameters for use in neuromodulation systems, such as a DBS, without recourse to any aggregation or common space analysis.

FIG. 6A illustrates an example embodiment of block diagram 600a using different targets to train learning models with imaging data using anatomical targets as input data, in accordance with one embodiment. Block diagram 600a includes two patient-specific segmentation images 602a and 608a.

For example, a first patient-specific segmentation image 602a includes a probe 606a with a region of interest 604a compared to a second patient-specific segmentation image 608a that includes a probe 612a with a second target nucleus 610a. By training learning models with imaging data, such as MRI imaging data, focused on different targets, such as different target nuclei or different ROI 604a and 610a, the machine learning (ML) model can identify the leads and anatomies associated with the different targets. While nuclei are used as examples in this embodiment, a person having ordinary skill in the art will understand the invention to include a variety of anatomical targets. For example, a region of interest (ROI), such as 604a and 610a, can be identified, defined, or wanted around one or more leads with one or more nuclei. Other ROIs can include a first target nucleus, or other anatomical features.

Examples of the disclosure include identifying similarity metrics among patients that are displaying (e.g., showing, finding, objectively identifying, subjectively feeling, etc.) similar improvements due to the DBS, where that improvement is presumed because of the DBS is doing something similar to each patient's brain. The effect of DBS begins at the local level (e.g., a region around the lead, region of interest (ROI), etc.), there is assumed to be one or more commonalities in the local region around the lead among or between these patients. Based on these commonalities, example embodiments can capture the similarities without recourse to a common atlas or space.

A key aspect of the disclosed technique is the identification and analysis of anatomical targets in medical imaging data such as MRI scans. Anatomical targets refer to specific structures, regions, or landmarks of interest relevant to a patient's condition or the implantation procedure. For instance, common anatomical targets for deep brain stimulation include the subthalamic nucleus (STN), globus pallidus interna (GPi), and ventral intermediate nucleus (VIM). These targets can be defined based on standard atlases or manually segmented from patient-specific MRI scans, which enable clear visualization of soft tissue structures. Once anatomical targets are identified in the imaging data, they can guide therapy planning and precise targeting of interventions such as electrode implantation surgery. Additionally, machine learning algorithms may be employed to automatically detect anatomical targets based on learned visual patterns. Therefore, the disclosed techniques leverage anatomical target information derived from medical imaging to optimize therapeutic parameters for an individual patient while minimizing reliance on broader atlas registration procedures.

FIG. 6B illustrates an example embodiment of block diagram 600b using different fields of view to train learning models with imaging data using windows about a lead as input data, in accordance with one embodiment.

Block diagram 600b includes three patient-specific segmentation 600b-1/600b-2/600b-3 images with three differently sized fields of view (FoV) 616b/622b/628b around each lead, respectively. For example, the first patient-specific segmentation image 600b-1 includes a FoV 616b that illustrates a close-up window about a probe 620b identifying a focal area or area of focus 618b about the lead (not shown). In the second example, the second patient-specific segmentation image 600b-2 includes a FoV 622b that illustrates a medium-sized window about a probe 626b identifying an area of focus 624b about the lead (not shown). In the third example, the third patient-specific segmentation image 600b-3 includes a FoV 628b that illustrates a large-sized window about a probe 632b identifying an area of focus 630b about the lead (not shown).

A key aspect of the disclosed technique is the use of localized fields of view surrounding implanted leads as input data to train machine learning models for predicting optimal deep brain stimulation parameters. Rather than analyzing whole brain scans, the models are trained on cropped regions or “windows” of imaging data centered around each patient's implanted leads. The window size is adjusted as a hyperparameter, with smaller windows focusing on local anatomy and larger windows providing more context. Additionally, multiple windows may be extracted around different lead contacts or from different imaging modalities. The lead-centered windows are then used to train convolutional neural networks, recurrent neural networks, or other machine learning architectures to identify anatomical patterns predictive of therapeutic outcomes. This localized field of view strategy provides relevant features to the models while avoiding extraneous data that would diminish predictive accuracy. By using lead-specific windows as model inputs, the disclosed technique enables data-efficient training for precise, patient-specific therapy optimization without requiring broader co-registration.

FIG. 6C illustrates an example embodiment of block diagram 600c using different achieved lead-to-target relationships to train learning models with imaging data as input data, in accordance with one embodiment.

In block diagram 600c, a first relationship 600c-1 illustrates a first lead-to-target relationship 636c based on the identified location of the patient's lead within the patient's brain. Block diagram 600c further illustrates a second relationship 600c-2 and a third relationship 600c-3 identifying different lead-to-target relationships 640c and 648c, respectively.

A key objective of the disclosed technique in FIG. 6C includes maximizing proximity between implanted DBS leads and the intended anatomical therapeutic targets, which are predetermined based on the patient's condition. For example, for disorders like Parkinson's disease, common targets are the subthalamic nucleus (STN) and globus pallidus interna (GPi). Precisely placing leads as close to targets as possible optimizes stimulation selectivity and minimizes side effects. Targeting accuracy can be evaluated by measuring the distance between lead contacts and the intended anatomical target identified on postoperative imaging, with typical targeting error ranging 1-3 mm for experienced DBS surgeons. Advanced techniques including microelectrode recording, test stimulation, and intraoperative MRI aid in confirming or refining lead position relative to targets. After implantation, the achieved lead/target relationship directly impacts parameter selection, as closer proximity allows therapeutic effects at lower amplitudes. Therefore, both surgical targeting precision and post-implantation programming are affected by the lead/target distance. The disclosed technique maximizes this proximity, thereby improving stimulation localization and clinical outcomes.

Precise targeting begins with thorough preoperative planning using high-resolution MRI and computational modeling. The target region is identified, and coordinates mapped to the stereotactic frame. During surgery, microelectrode recording provides electrophysiological confirmation of target structures. Test stimulation elicits clinical effects and verifies proximity. Adjustments are made iteratively based on this feedback to optimize lead placement. Frame-based, frameless, and intraoperative MRI-guided approaches aid accuracy. Robotic systems increase precision. During implantation, factors like brain shift after dura opening can cause deviation from planned trajectory. Ultrasound, CT, and intraoperative MRI help update for brain shift. Surgeons may angle multiple trajectories to triangulate targets. Lead contacts are positioned to span the target in the vertical axis, while avoiding adjacent structures. Multiple passes confirm vertical coverage intraoperatively. Postoperative imaging verifies coverage and proximity to the intended target. As such, surgical planning and techniques like microelectrode recording, test stimulation, brain shift compensation, robotic assistance, and confirmation imaging maximize targeting accuracy and optimal coverage of the therapeutic target region by implanted DBS leads.

As shown in block diagram 600c, maximizing proximity between implanted DBS leads and the anatomical therapeutic targets can be used as an input to train learning models with lead and anatomy. For example, input labels (e.g., electrode type, location, etc.) can be used to identify the lead, and feature labels can be used to identify the target in order to identify a lead/target relationship as input data.

Additional examples of the present disclosure enable prediction of stimulation parameters without the use of stimulation field models (SFMs), also referred to as volume of tissue activated (VTA) modeling, which involves creating computational models to determine the spread of electrical stimulation through brain tissue generated by deep brain stimulation (DBS). These SFM models aim to identify which neurons and neural pathways are activated by modeling the electric field produced by a given set of DBS parameters. By modeling the volume of activation, SFM can help predict and visualize the regions of the brain tissue that will be stimulated for a particular electrode placement and parameter setting. Users (e.g., surgeons, technicians, clinicians, etc.) can then leverage these models to guide selection of optimal DBS targets and associated stimulation parameters tailored to achieve desired therapeutic effects for a patient while avoiding side effects.

Example embodiments enable SFM to be replaced by data-driven machine learning techniques that could determine optimal DBS parameters directly from patient outcome data, without requiring explicit modeling of the stimulation fields. In additional examples, information available from the structural components of the neuroanatomical areas relevant to the DBS (e.g., those areas in the immediate vicinity of the lead) are integral to processes that form brain atlases, such as the standard brain. SFMs, for example, are modeled based on processes that synthesize these structural features. Examples of the system disclosed in FIGS. 6A-6C can directly use a machine learning model to learn structural properties of these brain areas to provide necessary information to predict the outcomes, while staying clear from the simplifying assumptions and/or noise inherent in aggregation processes.

According to one or all of examples disclosed in FIGS. 6A-6C, a variety of input details can be used to identify one or more of a variety of outputs, such as an intended target, a need for surgical revision, acute outcomes performance, or the like. Output from the machine learning models can provide additional information for use in predicting best stimulation parameters for new patients by including values or output data on a variety of neuromodulation information. For example, the proximity between implanted DBS leads and the intended anatomical targets directly impacts post-implantation programming and parameter selection. Closer lead/target alignment enables therapeutic effects to be achieved at lower amplitudes, thereby minimizing stimulation spread to adjacent structures and side effects. In contrast, increased lead/target distance may necessitate higher amplitudes to induce therapeutic effects, which risks current spread beyond the target and unintended stimulation. Ideal lead placement provides vertical coverage spanning the dorsal-ventral length of the target nucleus. Lateral targeting errors could result in stimulation of functional areas adjoining the target. Programming strategies including current steering and directional leads can help compensate for suboptimal lead/target alignment. However, if the distance is too great, surgical revision may be required to re-implant the leads closer to the target. Therefore, both the surgical targeting precision as well as the resulting lead/target distance play critical roles in determining optimal programming parameters and stimulation field shaping to maximize therapeutic benefits and minimize side effects.

In additional examples, the input into the learning models and/or the output of the trained learning models can be deployed to customer-facing software in order to further predict best stimulation parameters on new patients.

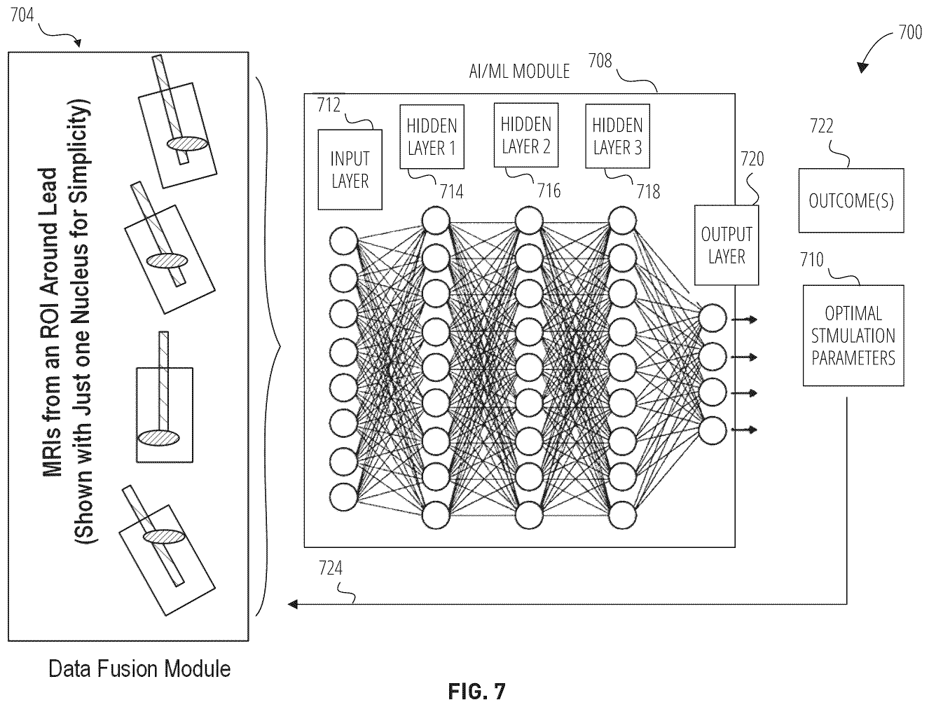

FIG. 7 is a block diagram 700, by way of example and not limitation, illustrating a data fusion module 704 used for training a network on imaging and/or other data in order to predict optimal parameter settings, in accordance with an example embodiment.

According to examples of the block diagram 700, systems can train one or more networks on imaging and other data to predict optimal (e.g., best) stimulation settings in order to predict optimal stimulation parameters on a new patient (e.g., patient of interest). For example, the data fusion module 704 can include MRIs from a region of interest (ROI) around one or more leads with one or more nuclei. Additional example embodiments can use an offline (e.g., simulated) algorithm-guided DBS-programming based on external sensor feedback (e.g., closed-loop programming evaluation using external response (CLOVER)) to streamline and facilitate programming of DBS using wearable feedback compared to individualized or aggregated programming.

Additional example embodiments can train a network on imaging and other data, as well as stimulation settings, based on the data fusion module 704 including MRIs from an ROI around a lead plus an electric field (e-field), where each voxel has a 4-dimensional object associated with it. In such an example, the implantable DBS system generates an e-field in the patient's brain tissue via the electrical stimulation delivered through the implanted electrode contacts. The e-field magnitude at any given point depends on factors such as, for example, the distance from the electrode contact, the conductivity of the surrounding tissue, and/or stimulation parameters like amplitude, pulse width, frequency, and the like. This induced electric potential causes modulated neural activity in the region surrounding the electrode which provides therapeutic effects. The volume of tissue activated (VTA) by the stimulation is defined by the area where the e-field is sufficient to modify neural firing. Patient-specific computational models can simulate the e-field and VTA based on the electrode configuration and stimulation settings. This modeling guides selection of optimal stimulation parameters to target the intended brain structures. In essence, the DBS system leverages optimized e-fields produced by programmed electrical stimulation to achieve desired therapeutic modulation of neurological activity.

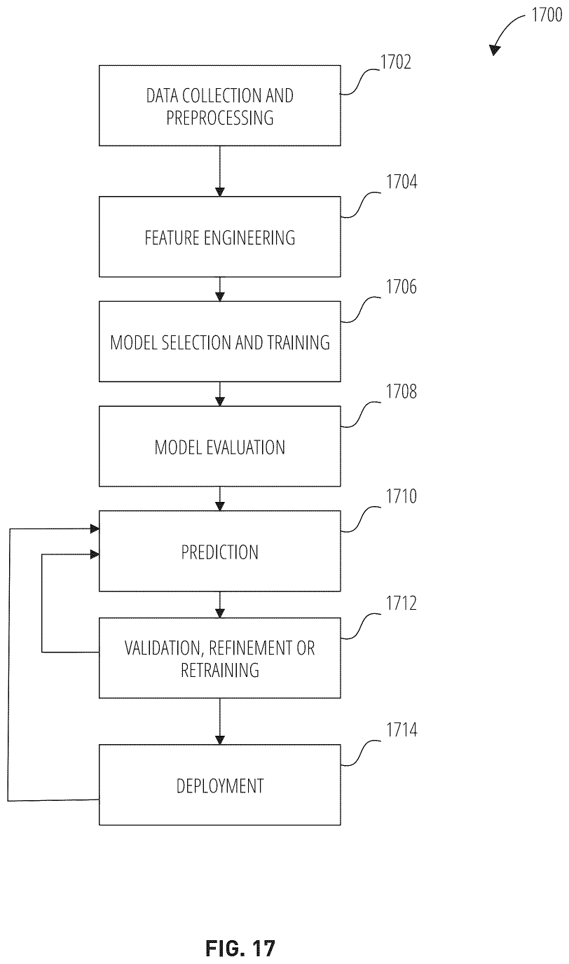

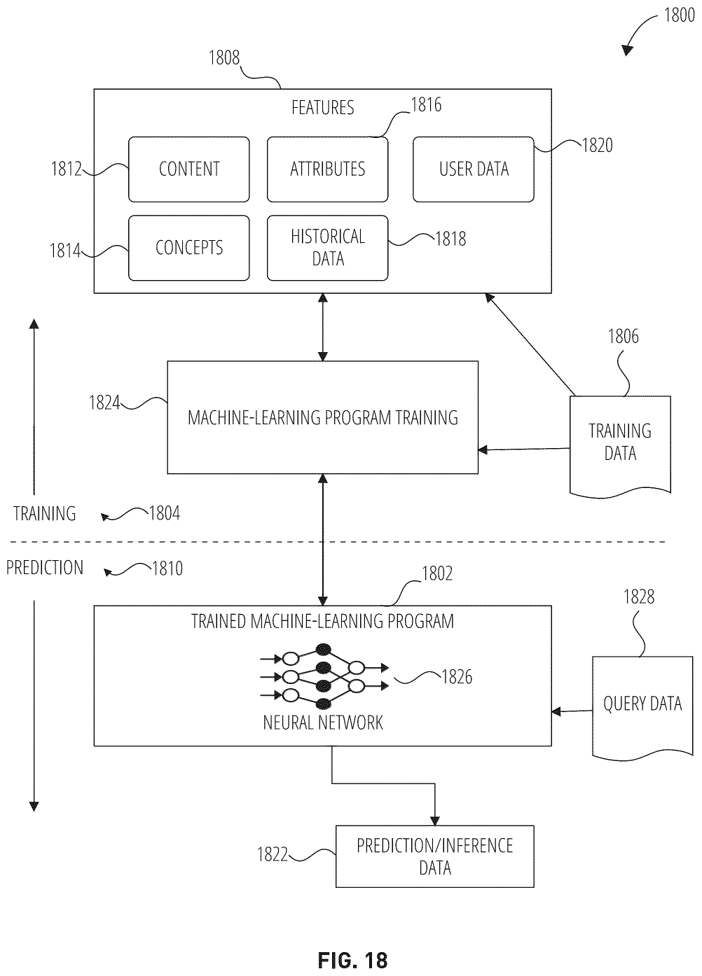

Returning to the block diagram 700, the data fusion module 704 can transmit or provide imaging data to an AI/ML module 708, which performs a variety of ML calculations in order to predict outcomes 722, such as optimal stimulation parameters 710 or other outputs. Broadly, the AI/ML module 708 may include machine learning that involves using one or more computer algorithms to automatically learn patterns and relationships in data provide from the data fusion module 704, potentially without the need for explicit programming. Machine learning algorithms can be divided into three main categories: supervised learning, unsupervised learning, and reinforcement learning. Example embodiments of machine learning are further described and depicted in connection with FIGS. 17 and 18.

According to some examples, the trained networks are able to precisely contour anatomical targets in new MRI scans (e.g., MRI scans of a new patient) based on learned patterns, such as textural patterns, spatial patterns, or the like. This eliminates reliance on manually defined atlases or templates. The machine learning approach thereby facilitates automated, adaptable, and highly accurate segmentation to precisely localize therapeutic targets in the brain for individual patients.

In some examples of the AI/ML module 708, a neural network may be generated during the training phase and implemented within the trained machine-learning program. The neural network can include a hierarchical (e.g., layered) organization of neurons, with each layer consisting of multiple neurons or nodes (illustrated with circles in the AI/ML module 708). Neurons in the input layer 712 receive the input data (e.g., data from the data fusion module 704), while neurons in the output layer 720 produce the final output of the network. Between the input layer 712 and the output layer(s) 720, there may be one or more hidden layers, such as hidden layer 1 714, hidden layer 2 716, and hidden layer 3 718, each consisting of multiple neurons. Each neuron in the neural network operationally computes a function, such as an activation function, which takes as input the weighted sum of the outputs of the neurons in the previous layer, as well as a bias term. The output of this function is then passed as input to the neurons in the next layer. If the output of the activation function exceeds a certain threshold, an output is communicated from that neuron (e.g., transmitting neuron) to a connected neuron (e.g., receiving neuron) in successive layers. The connections between neurons (not shown) have associated weights, which define the influence of the input from a transmitting neuron to a receiving neuron. During the training phase, these weights are adjusted by the learning algorithm to optimize the performance of the network. Different types of neural networks may use different activation functions and learning algorithms, affecting their performance on different tasks. The layered organization of neurons and the use of activation functions and weights enable neural networks to model complex relationships between inputs and outputs, and to generalize to new inputs that were not seen during training.

According to some example embodiments of the block diagram 700, training one or more networks to predict optimal settings without the need for a common space includes training a network to identify (e.g., learn) what features of imaging data are relevant in determining beneficial clinical effects, detrimental clinical effects, relevant anatomical data, or the like, such as learning to recognize a target (e.g., diSTN) in the brain. According to some examples, a network does not require segmentation of the brain, although segmentations can be included in addition to or instead of MRI images. According to some examples of training a network to predict best parameter settings, a network does not require SFMs or the like, although SFM data may be included, as well as using e-fields, second derivatives, or the like. According to some examples, networks can be hierarchical (e.g., level 1 learning best contact level, level 2 learning best contact fraction, level 3 learning best amplitude, or the like). In additional examples, networks can include data other than MRI data and/or other than imaging data.

In a prediction phase, the trained machine-learning program uses the features for analyzing query data to generate inferences, outcomes, or predictions, as examples of a prediction/inference data. For example, during prediction phase, the trained machine-learning program generates an output layer 720. Query data (not shown) is provided as an input to the trained machine-learning program, and the trained machine-learning program generates the prediction/inference data as output, responsive to receipt of the query data.

According to additional example embodiments, the AI/ML module 708 can receive additional input data from one or more modules besides the data fusion module 704; for example, an objective data module, a subjective data module, or other data source for use as additional data to be processed by the AI/ML module 708. In some examples of the data fusion module 704, stimulation settings can be fused with imaging data according to a variety of methodologies. For example, stimulation settings can be fused with imaging data by multiplying the e-field with the MRI intensity, or the e-field along the fiber direction in the voxel with the MRI intensity, or other such methods. In some examples, SFMs can be used as a simple fusion method (e.g., to binarize the MRI into relevant and/or irrelevant data). In some examples of the data fusion module 704, additional types of data may be included, such as electrophysiology data, patient state data, or the like.

For example, the objective data module (not show), can include objective data, which is data that can be observed using human senses and can be obtained from a measurement or direct observation. Objective data can be measured by a sensor and can be provided via user input when the user has access to objectively determined information. Examples of objective data can include physiological parameter data, therapy data, device data, environment data, and/or additional objective data (e.g., health related data). By way of example and not limitation, physiological parameter data can include data such as: heart rate, blood pressure, respiration rate, activity, posture, electromyograms (EMGs), neural responses such as evoked compound action potentials (ECAPs), glucose measurements, oxygen levels body temperature, oxygen saturation, and gait. By way of example and not limitation, therapy data can include neuromodulation programs, therapy on/off schedule, dosing, neuromodulation parameters such as waveform, frequency, amplitude, pulse width, period, therapy usage and therapy type. By way of example and not limitation, device data can include battery information (voltage, charge state, charging history if rechargeable), impedance data, faults, device model, lead models, MRI status, Bluetooth connection logs, connection history with Clinician's Programmer (CP). By way of example and not limitation, environment data can include temperature, air quality, pressure, location, altitude, sunny, cloudy, precipitation, etc. By way of example, additional data can include healthcare-related data (e.g., menstrual cycles, surgeries, procedures, acute conditions, other non-pain chronic conditions, etc.), lifestyle-related data (e.g., relationship problems, financial problems, at-home stressors, at-work stressors, etc.), and the like.

For example, a subjective data module (not shown) can include subjective data that includes information received from one or more patients. For example, the patient's quantification of pain is a subjective data. Subjective data can generally involve user-inputted data. Examples of subjective data include questions with free text answer(s), multiple choice questions, question tree(s), and/or additional subjective data. Other data can be stored and/or transferred, including detected event(s), contextual data (e.g., context(s)) for other collected data and/or event(s), and a clock (e.g., time) such as can be used to provide a time stamp associated with the retrieved data. The event(s), context(s), and time can be detected by the system or can be provided via user input and received by the AI/ML module 708 as input data.

The collected data (whether from the data fusion module 704 or other data sources) can be processed at a data processing component of the AI/ML module 708 or another module (not shown). The data processing can occur in a medical device or a patient device such as a phone, tablet, or remote control, or can occur in a remote data receiving system. The data processing can include one or more model(s). The model(s) can be used to determine how the patient data is used to determine commonalities (e.g., common issues, beneficial clinical effects, detrimental clinical effects, etc.) for which prediction outcomes for all stimulation settings of a patient of interest (e.g., one or more new patients) can be based upon. Machine learning (or other artificial intelligence) can be implemented on the collected data to develop or refine the model(s). The data processing can include data imputation such as can be used to prevent missing data from introducing bias into the model(s) or machine learning. According to some examples, the output data, such as data from the output layer 720, can be provided back to the data fusion module 704 as new input data 724.

A key aspect of the disclosed technique is the application of machine learning algorithms, including by way of example and not limitation, convolutional neural networks (CNNs), recurrent neural networks (RNNs), generative adversarial networks (GANs), or the like to automatically detect and delineate anatomical targets in medical images, such as MRI scans. These artificial intelligence models are trained on datasets of MRI images (or other imaging or non-imaging data) along with corresponding manual segmentations or weak labels marking the anatomical targets. A variety of training strategies, including supervised, semi-supervised, weakly-supervised, and data augmentation techniques, are employed to optimize model performance used by the AI/ML module 708. Architectural nuances such as encoder-decoder connections in U-Nets and long short-term memory (LSTM) units in RNNs further refine model accuracy according to some examples. Ensembling multiple models and post-processing with mathematical morphology operations help improve segmentation precision in additional examples.

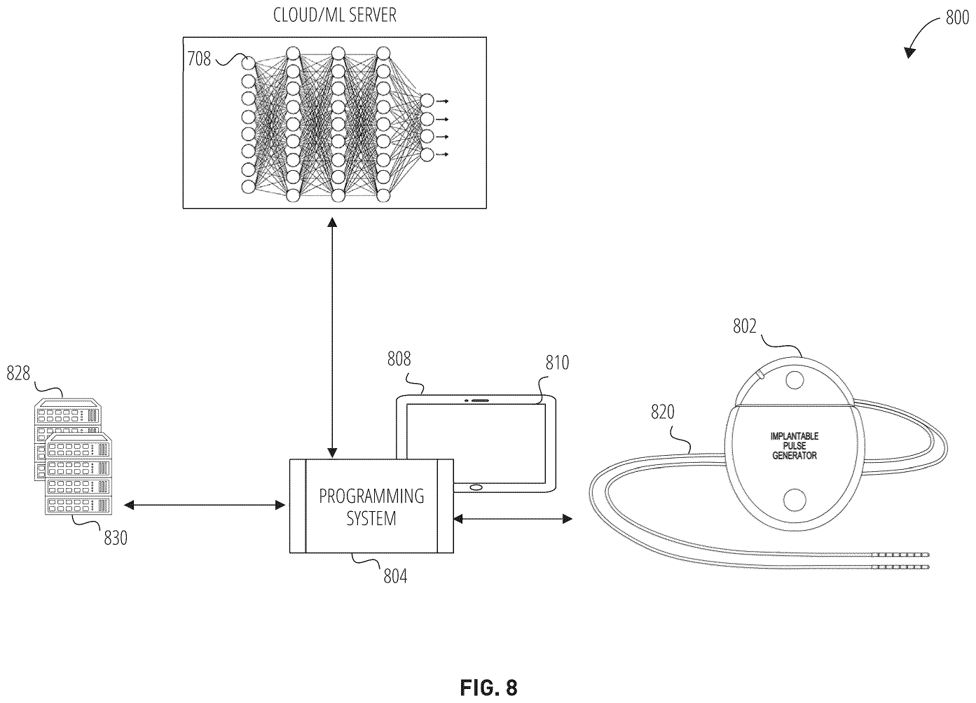

FIG. 8 illustrates an embodiment of a block diagram depicting a neurostimulation system 800 communicatively coupled to various databases 828/830 via a communication network (not shown), in accordance with one example embodiment.

The neurostimulation system 800 can include a stimulator, such as an implantable pulse generator 802, coupled to leads of a lead system 820 and interconnected to an external programming system 804, which can be implemented in an external programming device that includes stimulation programming circuitry (not shown). For example, FIG. 8 illustrates an embodiment the neurostimulation system 800 communicatively coupled to a first database 828 and a second database 830 via a telecommunication system. The neurostimulation system 800 can include implantable stimulator 802 communicatively coupled to the programming system 804, which can be implemented in an external programming device (not shown) and includes stimulation programming circuit (not shown).

A stimulation programming circuit can be used in the process of determining the settings of implantable stimulator including the stimulation configuration for the patient and/or the process of positioning lead(s) 820 in the patient (e.g., a patient of interest). Databases 828/830 can include any number and types of databases containing the aggregate data collected from the patient as well as portions of the patient-specific data (e.g., the patient's medical records, etc.). Each database 828/830 can belong to a user, a group of users, or an organization participating in the care of patients receiving therapies including neurostimulation and include patient data as used throughout this application (e.g., patient imaging data).

A telecommunication system can provide for communications between the external programming system 804 and databases 828/830 using one or more wireless communication links or networks such as the Internet, intranets, cellular networks, Bluetooth, or the like. Additionally, when needed, data can also be transferred from each of databases 828/830 to an external programming device using portable data storage media. In various embodiments, external programming device can be implemented as one or more devices, such as being implemented in a mobile device 808, such as a computer or mobile device, or the like, which can include a user interface 810.

In various embodiments, aggregate data stored in one or more databases 828/830 or other data lakes can include data collected from each patient of the patient population directly (e.g., signals sensed from each patient, answers to questions presented to each patient, etc.) and/or indirectly (e.g., operational information extracted from each device used to treat the patients, information extracted from each patient's medical record, etc.). The patient-specific data can include data collected from the patient directly (e.g., signals sensed from the patient and/or answers to questions presented to the patient) and/or indirectly (e.g., operational information extracted from a device used to treat the patient and/or information extracted from the patient's medical record).

Returning to the system architecture of FIG. 8, the neurostimulation system 800 illustrates a light deployment as ML models, such as ML models of the AI/ML module 708, only require large processing power for training and not necessarily for prediction of optimal parameter settings according to examples of the present disclosure. The programming system 804 is operatively connected with the implantable device 802 in order to provide (e.g., deliver) neurostimulation energy, such as in the form of electrical pulses, to the one or more neural targets though electrodes 820. The delivery of the neurostimulation is controlled by using a plurality of stimulation parameters, such as stimulation parameters specifying a pattern of the electrical pulses and a selection of electrodes through which each of the electrical pulses is delivered.

In various embodiments, at least one or more parameters of the plurality of stimulation parameters are selected or programmable by a clinical user, such as a physician or other caregiver who treats the patient using the neurostimulation system 800; however, some of the parameters may also be provided in connection with closed-loop programming logic and adjustment. In various embodiments, programming device 808 includes the user interface 810 (e.g., a user interface embodied by a graphical, text, voice, hardware-based user interface, or the like) that allows the user to set and/or adjust values of the user-programmable parameters by creating, editing, loading, and removing programs that include parameter combinations such as patterns and waveforms. These adjustments may also include changing and editing values for the user-programmable parameters or sets of the user-programmable parameters individually (including values set in response to a therapy efficacy indication). Such waveforms may include, for example, the waveform of a pattern of neurostimulation pulses to be delivered to the patient as well as individual waveforms that are used as building blocks of the pattern of neurostimulation pulses. Examples of such individual waveforms include pulses, pulse groups, and groups of pulse groups. The program and respective sets of parameters may also define an electrode selection specific to each individually defined waveform.

In additional example embodiments, the databases 828/830 can include other healthcare related data source(s) configured for use to collect healthcare-related data for characterization of parameterizing techniques for identifying similarity metrics without the need for warping tools or anatomical segmentation software. By way of example, an embodiment of the neurostimulation system 800 or a component thereof is configured to collect and analyze healthcare-related data in connection with the use of neurostimulation programs and closed-loop neurostimulation programming. For example, the data inputs can be processed to generate analysis data (not shown), which represents a transformed or refined version of data based on actual patient usage, patient feedback (e.g., feedback that indicates what settings have been effective or have not been effective), patient parameter inputs, similarity metrics, stimulation settings, or the like. The analysis data can include additional data processing including, for example, parameter data, cluster solution data, accellerometer data, ECAP data, and additional imaging data.

Additional examples of the databases 828/830 may provide additional data, such as patient data, medical device data, patient environmental data, therapy data, patient lifestyle data, and/or other imaging data, particularly in specialized neurostimulation programming environments. Other healthcare-related data source(s) may include patient data received via a provider's server that stores patient health records. For example, patients may use a patient portal to access their health records such as test results, doctor notes, prescriptions, and the like. Other healthcare-related data sources may include applications on a patient's smartphone or other computing device, or the data on a server accessed by those applications. In another example, an application on a phone or patient's device may include or may be configured to access environmental data such as weather data and air quality information or location elevation data such as may be determined using cellular networks and/or a global positioning system (GPS). Weather data may include, but is not limited to, barometric pressure, temperature, sunny or cloud cover, wind speed, and the like. By way of example and not limitation, this type of data may include heart rate, blood pressure, weight, and the like collected by the patient in their home. In additional examples, this type of data may include ECAP factors, such as amplitude, detection threshold, perception level, linearity, stimulation parameters, electrode/lead/paddle locations, neural activation, or the like. The amplitude or size of the recorded ECAP signal provides information about the number of nerve fibers activated by the electrical stimulation. Larger ECAP amplitudes indicate more nerve fiber recruitment. ECAPs exhibit a detection threshold below which they cannot be observed or recorded, even if nerve activation is occurring. This is a technological limitation, not a physiological limitation. For example, in some examples, ECAPs may only be recordable at perception levels and above in patients. In additional examples, no or few ECAP signals can be detected during sub-perception stimulation. Once above the detection threshold, ECAP amplitude grows approximately linearly with increasing stimulation intensity. Factors like stimulation amplitude, pulse width, frequency affect the ECAP response and must be optimized to target fibers. The recording electrode location impacts the ECAP signal and must be carefully placed in order to detect responses. According to varying examples of the present disclosure, combinations of data stored, organized, and correlated in the databases 828/830 can be used in a variety of embodiments.

The programming system 804 and/or the AI/ML module 708 may be implemented at one or more server(s) or other systems remotely located from the patient. The neurostimulation system 800 may use various network protocols to communicate and transfer data through one or more networks, which may include the Internet. The neurostimulation system 800 can include at least one processor (not shown) configured to execute instructions stored in memory (e.g., depicted as processor(s)/memory) to generate or evaluate data outputs, to obtain or evaluate data inputs, and to perform data processing on both inputs and outputs and accompanying training data of the AI/ML module 708. Further, the external system(s) may be configured to receive data from an associated medical device(s) and/or receive data from other healthcare-related data source(s), and then transfer the data through the network(s) to the data receiving system(s).

Additional examples may include implantable devices, such as the implantable device 802, configured to sense nerve activity such as evoked compound action potentials (ECAPs), which can identify and record nerve conduction, feeling, activity, and other patient monitors. The implantable device may be configured to sense nerve activity (e.g., ECAPs) using the electrodes, and may be further configured to evaluate sensing-capable electrodes.

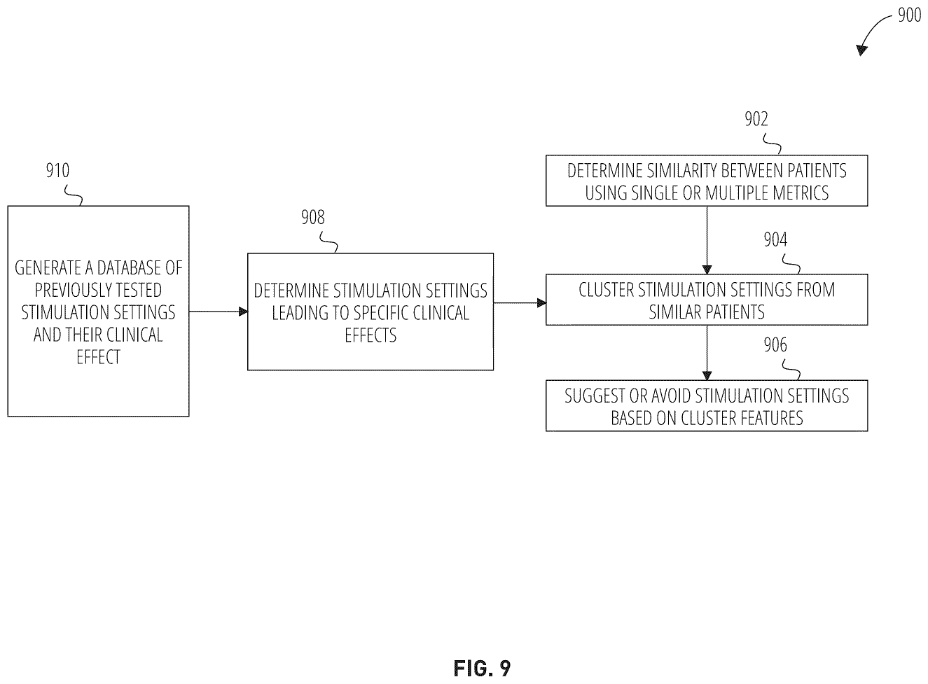

FIG. 9 illustrates a flowchart showing one example of a routine 900 (e.g., a process flow, processing method, etc.) for automatically determining stimulation settings based on similarity metrics, in accordance with example embodiments of the present disclosure.

The embodiment of the routine 900 can be implemented by a system or device for use to determine deep brain stimulation (DBS) stimulation setting optimization according to parameter values that lead to beneficial clinical effects or detrimental clinical effects. For example, the routine 900 can be embodied by electronic operations performed by one or more computing systems or devices (including those at a network-accessible remote service) that are specially programmed to automatically determine stimulation settings based on similarity metrics of a plurality of patients using previously gathered and organized data. In specific examples, the operations of the routine 900 can be implemented through the systems and data flows depicted throughout the disclosure, at a single entity or at multiple locations. For example, the routine 900 can be performed by the neuromodulation system 115a or a component thereof as described and depicted in connection with FIG. 1A or the neurostimulation system 800 or a component thereof as described and depicted in connection with FIG. 8.

In block 910, routine 900 generates a database of previously tested stimulation settings and their clinical effect. In block 908, routine 900 determines stimulation settings leading to specific clinical effects. In block 902, routine 900 determines similarity between patients using single or multiple metrics. In block 904, routine 900 clusters stimulation settings from similar patients. In block 906, routine 900 suggests or avoids stimulation settings based on cluster features.

According to some example embodiments of the routine 900, the routine 900 may begin at block 910. According to other example embodiments of the routine 900, the routine 900 may begin at block 902. According to further examples of the routine 900, the routine may simultaneously or near-simultaneously begin at both block 902 and block 910.

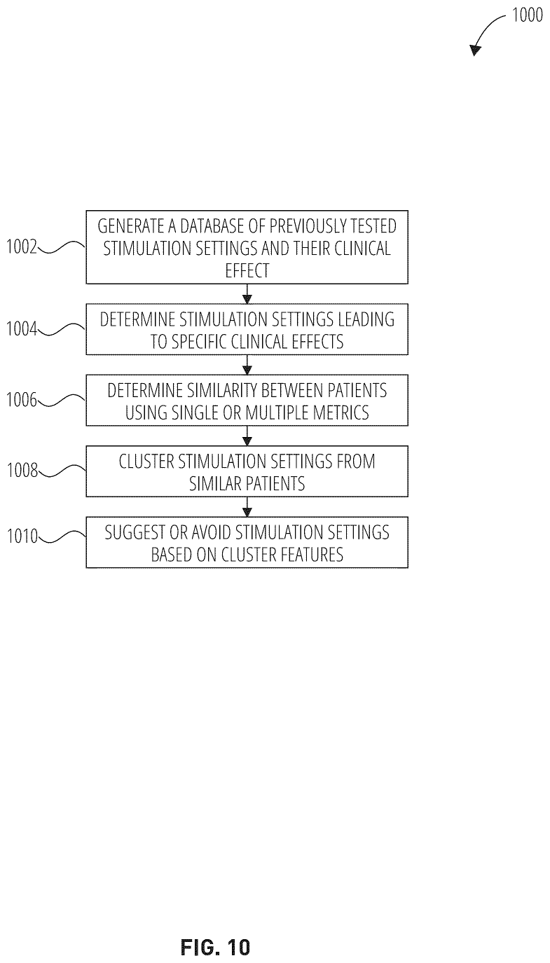

FIG. 10 is a flowchart illustrating a routine 1000 of machine learning based automated deep brain stimulation (DBS) programming, in accordance with one embodiment.

The embodiment of the routine 1000 can be implemented by a system or device for use to determine DBS stimulation setting optimization according to parameter values that lead to beneficial clinical effects or detrimental clinical effects. For example, the routine 1000 can be embodied by electronic operations performed by one or more computing systems or devices (including those at a network-accessible remote service) that are specially programmed to automatically determine stimulation settings based on similarity metrics of a plurality of patients using previously gathered and organized data. In specific examples, the operations of the routine 1000 can be implemented through the systems and data flows depicted throughout the disclosure, at a single entity, or at multiple locations. For example, the routine 1000 can be performed by the neuromodulation system 115a or a component thereof as described and depicted in connection with FIG. 1A or the neurostimulation system 800 or a component thereof as described and depicted in connection with FIG. 8.

In block 1002, routine 1000 generates a database of previously tested stimulation settings and their clinical effect. In block 1004, routine 1000 determines stimulation settings leading to specific clinical effects. In block 1006, routine 1000 determines similarity between patients using single or multiple metrics. In block 1008, routine 1000 clusters stimulation settings from similar patients. In block 1010, routine 1000 suggests or avoids stimulation settings based on cluster features.

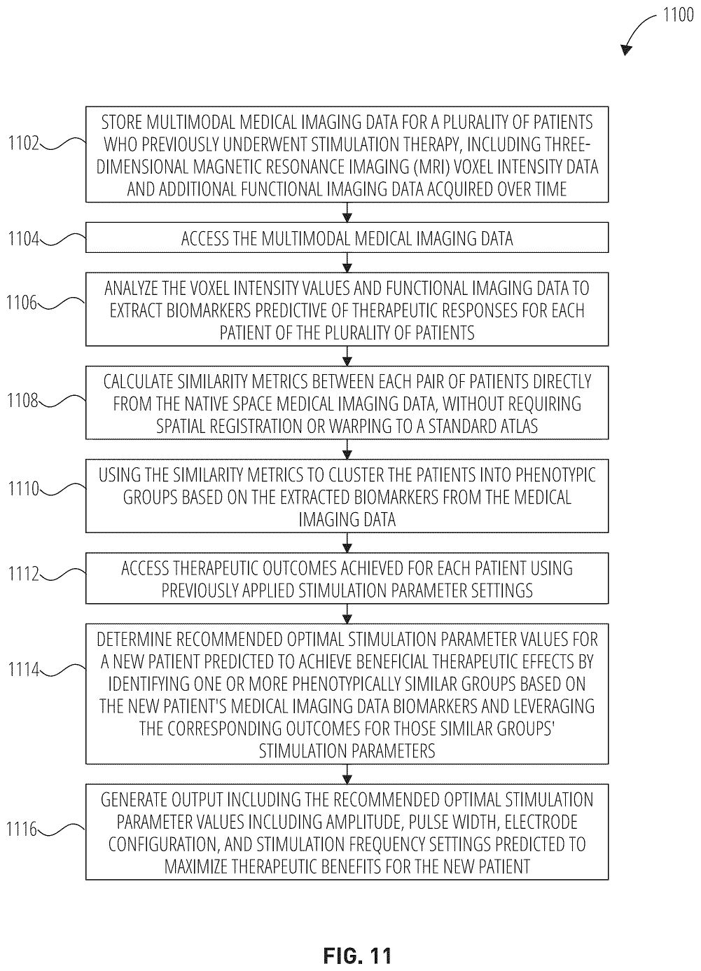

FIG. 11 illustrates a routine 1100 showing a method of predicting stimulation settings utilizing information from voxel intensities from imaging data in areas adjacent to implanted leads from a patient's native space imaging, in accordance with example embodiments of the present disclosure.

The embodiment of routine 1100 can be implemented by a system or device for use to predict stimulation parameters for a patient of interest according to imaging data from a plurality of patients. For example, the routine 1100 can be embodied by electronic operations performed by one or more computing systems or devices (including those at a network-accessible remote service) that are specially programmed to implement machine learning based on automated neurostimulation programming according to example embodiments of the present disclosure. In specific examples, the operations of the routine 1100 can be implemented through the systems and data flows depicted throughout the disclosure, at a single entity, or at multiple locations. For example, the routine 1100 can be performed by the neuromodulation system 115a or a component thereof as described and depicted in connection with FIG. 1A or the neurostimulation system 800 or a component thereof as described and depicted in connection with FIG. 8.

In block 1102, routine 1100 stores multimodal medical imaging data for a plurality of patients who previously underwent stimulation therapy, including three-dimensional magnetic resonance imaging (MRI) voxel intensity data and additional functional imaging data acquired over time. In block 1104, routine 1100 accesses the multimodal medical imaging data. In block 1106, routine 1100 analyzes the voxel intensity values and functional imaging data to extract biomarkers predictive of therapeutic responses for each patient of the plurality of patients.

In block 1108, routine 1100 calculates similarity metrics between each pair of patients directly from the native space medical imaging data, without requiring spatial registration or warping to a standard atlas. In block 1110, routine 1100 using the similarity metrics to cluster the patients into phenotypic groups based on the extracted biomarkers from the medical imaging data. In block 1112, routine 1100 accesses therapeutic outcomes achieved for each patient using previously applied stimulation parameter settings.

In block 1114, routine 1100 determines recommended optimal stimulation parameter values for a new patient predicted to achieve beneficial therapeutic effects by identifying one or more phenotypically similar groups based on the new patient's medical imaging data biomarkers and leveraging the corresponding outcomes for those similar groups' stimulation parameters. In block 1116, routine 1100 generates output including the recommended optimal stimulation parameter values including amplitude, pulse width, electrode configuration, and stimulation frequency settings predicted to maximize therapeutic benefits for the new patient.

According to additional examples of the present invention, embodiments allow selecting and applying one or more phenotypic criteria to stratify the patient population into subgroups exhibiting differential therapeutic responses to neurostimulation. Examples of phenotypic stratification criteria include anatomical traits from medical imaging such as subcortical volumes or white matter integrity, genetic markers such as single nucleotide polymorphisms correlating with disease subtypes, symptom profiles categorizing patients based on presence and severity of specific symptoms, disease progression stage and severity, presence of comorbidities like depression, demographic factors like age and gender, baseline levels of relevant physiological biomarkers prior to therapy, degree of cognitive/neuropsychological impairment, variability in response to medications, family history and environmental factors indicating hereditary versus sporadic subtypes, sleep quality metrics, and more, including the lack of or absence any of such phenotypic criteria. Both single and multiple criteria could be utilized for stratification. The choice of optimal criteria depends on those exhibiting the strongest correlations with differential therapeutic responses across the resulting subgroups. Applying neurostimulation parameters tailored to a patient's phenotypic stratum enables personalized, precision therapy compared to a one-size-fits-all approach.

FIG. 12A illustrates, by way of example, a block diagram of an embodiment of a computing system 1201a implementing neurostimulation programming circuitry 1206a to cause programming of an implantable electrical neurostimulation device, for accomplishing the therapy objectives in a human subject based on a trained programming model as discussed herein.

The system 1201a may be operated by a clinician, a patient, a caregiver, a medical facility, a research institution, a medical device manufacturer or distributor, and embodied in a number of different computing platforms. The system 1201a may be a remote-control device, patient programmer device, program modeling system, or other external device, including a regulated device used to directly implement programming commands and modification with a neurostimulation device. In some examples, the system 1201a may be a networked device connected via a network (or combination of networks) to a computing system operating a user interface computing system using a communication interface 1208a. The network may include local, short-range, or long-range networks, such as Bluetooth, cellular, IEEE 802.11 (Wi-Fi), or other wired or wireless networks.

The system 1201a includes a processor 1202a and a memory 1204a, which can be optionally included as part of neurostimulation stimulation programming circuitry 1206a. The processor 1202a may be any single processor or group of processors that act cooperatively. The memory 1204a may be any type of memory, including volatile or non-volatile memory. The memory 1204a may include instructions, which when executed by the processor 1202a, cause the processor 1202a to implement the features of the stimulation programming circuitry 1206a (e.g., neurostimulation circuitry). Thus, the electronic operations in the system 1200a may be performed by the processor 1202a or the stimulation programming circuitry 1206a.

The processor 1202a or circuitry 1206a may directly or indirectly implement neurostimulation operations including the use of neurostimulation device programming based on a trained programming model. The processor 1202a or circuitry 1206a may further provide data and commands to assist the processing and implementation of the programming using communication interface 1208a or a stimulation device interface 1210a (e.g., neurostimulation interface). It will be understood that the processor 1202a or circuitry 1206a may also implement other aspects of the device data processing or device programming functionality described above.

FIG. 12B illustrates, by way of example, a block diagram of an embodiment of a computing system 1201b for performing automatic determination of stimulation settings based on one or more similarity metrics and/or for performing machine learning based automated DBS programming, in connection with the data processing operations discussed above.

The system 1201b may be integrated with or coupled to a computing device, a remote-control device, patient programmer device, clinician programmer device, program modeling system, or other external device, deployed with neurostimulation treatment. In some examples, the system 1201b may be a networked device (server) connected via a network (or combination of networks), which communicates to one or more devices (e.g., clients) via a user interface 1210b using a communication interface 1208b (e.g., communication hardware which implements software network interfaces and services). The network may include local, short-range, or long-range networks, such as Bluetooth, cellular, IEEE 802.11 (Wi-Fi), or other wired or wireless networks.

The system 1201b includes a processor 1202b and a memory 1204b, which can be optionally included as part of a stimulation programming circuitry 1206b (e.g., user input/output data processing circuitry). The processor 1202b may be any single processor or group of processors that act cooperatively. The memory 1204b may be any type of memory, including volatile or non-volatile memory. The memory 1204b may include instructions, which when executed by the processor 1202b, cause the processor 1202b to implement data processing, or to enable other features of the user input/output data processing stimulation programming circuitry 1206b. Thus, electronic operations in the system 1201b may be performed by the processor 1202b or the circuitry 1206b.

For example, the processor 1202b or the circuitry 1206b may implement any of the features of the routine 900 (e.g., method) to suggest or avoid stimulation settings based on cluster features. It will be understood that the processor 1202b or the circuitry 1206b may also implement aspects of the logic and processing described above, for use in various forms of open-loop, closed-loop, partially-closed-loop device programming or related device actions.

FIG. 13A illustrates a block diagram of a neurostimulation system 1300a of a patient system 1310b, and examples of devices that may make up components of the patient system, in accordance with some example embodiments.

For example, FIG. 13A illustrates an embodiment of the neurostimulation system 1300a. System 1300a includes electrodes 1322a, a stimulation device 1320a, and a programming device 1316a. Electrodes 1322a (e.g., paddles, leads, etc.) are configured to be placed on or near one or more neural targets in a patient. Stimulation device 1320a is configured to be electrically connected to electrodes 1322a and deliver neurostimulation energy, such as in the form of electrical pulses, to the one or more neural targets though electrodes 1322a. The delivery of the neurostimulation is controlled by using a plurality of stimulation parameters, such as stimulation parameters specifying a pattern of the electrical pulses and a selection of electrodes through which each of the electrical pulses is delivered.

In various embodiments, at least some parameters of the plurality of stimulation parameters are selected or programmable by a clinical user, such as a physician or other caregiver who treats the patient using system 1300a; however, some of the parameters may also be provided in connection with closed-loop programming logic and adjustment. Other embodiments may include open-loop programming and adjustment or other programming logic to identify one or more parameters. Programming device 1316a provides the user with accessibility to implement, change, or modify the programmable parameters. In various embodiments, programming device 1316a is configured to be communicatively coupled to stimulation device 1320a via a wired or wireless link.

In various embodiments, programming device 1316a includes a user interface 1318a (e.g., a user interface embodied by a graphical, text, voice, hardware-based user interface, etc.) that allows the user to set and/or adjust values of the user-programmable parameters by creating, editing, loading, and removing programs that include parameter combinations such as patterns and waveforms. These adjustments may also include changing and editing values for the user-programmable parameters or sets of the user-programmable parameters individually (including values set in response to a therapy efficacy indication). Such waveforms may include, for example, the waveform of a pattern of neurostimulation pulses to be delivered to the patient as well as individual waveforms that are used as building blocks of the pattern of neurostimulation pulses. Examples of such individual waveforms include pulses, pulse groups, and groups of pulse groups. The program and respective sets of parameters may also define an electrode selection specific to each individually defined waveform.

The present approaches further provide examples of an evaluation system 1312a, such as a data analysis system, which is used to adapt, modify, start, stop, monitor, or identify a neurostimulation treatment with stimulation device 1320a. This evaluation system 1312a initiates an action related to the neurostimulation treatment based on text analysis performed on input 1314a (e.g., input text). This input 1314a can be directly collected from the patient and analyzed by the evaluation system 1312a, to then cause a programming effect in the programming device 1316a, and the stimulation device 1320a, and the neurostimulation treatment provided by the electrodes 1322a.

A user (e.g., the patient, clinician, device representative, etc.) can provide parameter inputs to the evaluation system 1312a, which are used to select, load, modify, implement, measure, analyze, monitor, and/or evaluate one or more parameters of a defined program for neurostimulation treatment that is implemented by the stimulation device 1320a, or the operation of the stimulation device 1320a. This evaluation can be based on a combination of natural language processing, sentiment analysis, rules, and other operational or treatment objectives that are identified. Various logic, machine learning models, and/or algorithms can then determine an appropriate action to take based on the state of the patient, including but not limited to: a program or parameter change or recommendation to produce an improvement for a treatment objective (such as to address pain, increase mobility, reduce sleep disruption, and the like); diagnostic or remedial actions on the stimulation device 1320a; data logging or alerts to the patient or a clinician associated with the patient; and the like.

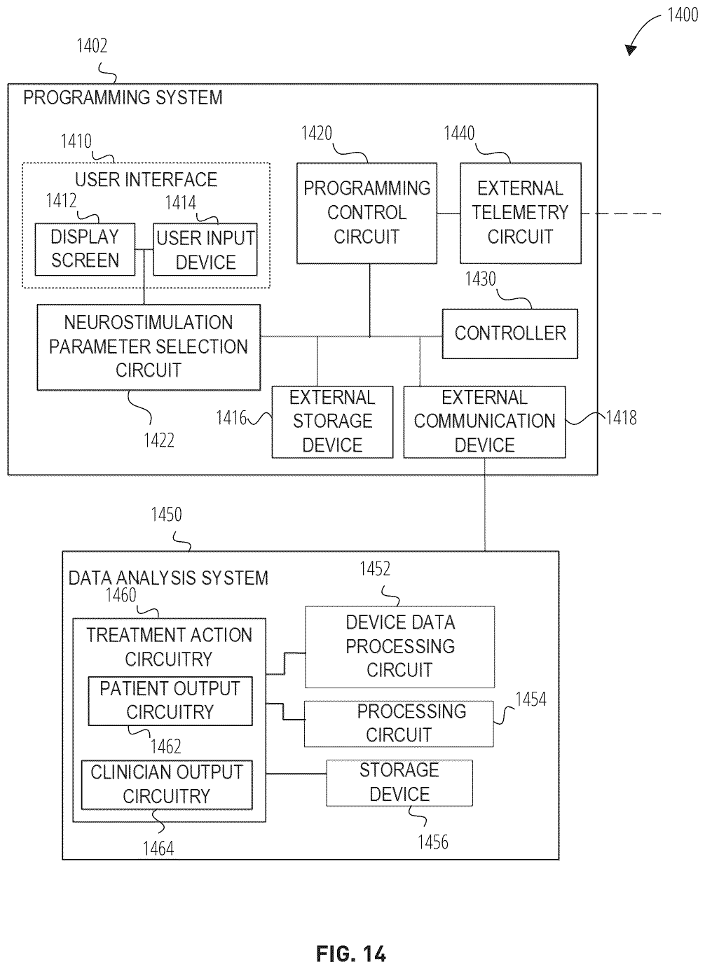

Example parameters that can be implemented by a selected neurostimulation program include, but are not limited to the following: amplitude, pulse width, frequency, duration, total charge injected per unit time, cycling (e.g., on/off time), pulse shape, number of phases, phase order, interphase time, charge balance, ramping, as well as spatial variance (e.g., electrode configuration changes over time). As detailed in FIG. 14, a controller, e.g., controller 1430 of FIG. 14, can implement program(s) and parameter setting(s) to affect a specific neurostimulation waveform, pattern, or energy output, using a program, or setting in storage (e.g., external storage device 1416 of FIG. 14), or using settings communicated via an external communication device 1418 of FIG. 14 corresponding to the selected program. The implementation of such program(s) or setting(s) may further define a therapy strength and treatment type corresponding to a specific pulse group, or a specific group of pulse groups, based on the specific programs or settings. The evaluation system 1312a and the evaluation of the input 1314a provides a mechanism to determine the effectiveness of such programs or settings, and to identify issues and provide remediation for ineffective programs or settings, offer suggestions or recommendations for new or updated programs and/or settings, or even to automatically change programs or settings.

Portions of the evaluation system 1312a, the stimulation device 1320a (e.g., implantable medical device, wearable device, etc.), or the programming device 1316a can be implemented using hardware, software, or any combination of hardware and software. Portions of the stimulation device 1320a or the programming device 1316a can be implemented using an application-specific circuit that can be constructed or configured to perform one or more particular functions or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. The system 1300a could also include a subcutaneous medical device (e.g., subcutaneous implantable cardioverter-defibrillator (S-ICD), subcutaneous diagnostic device, wearable medical devices (e.g., patch-based sensing device), or other external medical devices.

FIG. 13B illustrates a block diagram 1300b of a patient system 1310b, and examples of devices that may make up components of the patient system, in accordance with some example embodiments. The patient system 1310b may include component(s) that act on the patient such as deliver a therapy to the patient, component(s) used to sense a condition, status or environment of the patient, and component(s) enabling the patient to interface with the patient system.

For example, the illustrated patient system 1310b may include sensor(s) 1301b to sense patient parameter(s). Examples of sensors may include, but not limited to, neural activity, muscle movement, patient activity, patient posture, patient location, breathing, heart rate, blood pressure, temperature, analyte sensors and the like. The illustrated patient system 1310b may include a therapy-delivering device such a neuromodulator 1302b configured to deliver a neuromodulation therapy such as a DBS, SCS, PNS, FES, transcutaneous electrical nerve stimulation (TENS), or other therapy. Those of ordinary skill in the art would understand, upon reading and comprehending the present disclosure, how to apply the present subject matter to other therapies, such as but not limited to cardiac rhythm therapies or drug pump therapies.

The illustrated patient system 1310b may also include patient device(s) 1303b used by the patient to interface with the patient system. These device(s) may include sensor(s) (e.g., accelerometer, temperature sensor, heart rate sensor, etc.) and/or can be used to receive patient feedback such as responses to questions or free text responses. These device(s) may include interfaces used to interact with the therapy delivery. Examples of patient device(s) 1303b include, but are not limited to, a patient remote control 1304b, a wearable device(s) such as a watch 1305b, or a phone or tablet 1306b, or another personal device or additional input 1307b.

FIG. 14 illustrates a block diagram 1400 of a programming system 1402 used as part of an implantable neurostimulation system, such as the external system 114a as described and depicted in connection with FIG. 1, with the programming system 1402 configured to send and receive device data (e.g., commands, parameters, program selections, information), in accordance with example embodiments. FIG. 14 also illustrates an embodiment of a data analysis computing system 1450, communicatively coupled to the programming system 1402, with the data analysis computing system 1450 used to perform data analysis on freeform text and device data (or other types of data) in connection with neurostimulation treatment by the implantable neurostimulation system.

The programming system 1402 represents an embodiment of the programming device 1316a of FIG. 13A, and includes an external telemetry circuit 1440, an external storage device 1416, a programming control circuit 1420, a user interface (UI) 1410 device, a controller 1430, and an external communication device 1418, to effect programming of a connected neurostimulation device. The operation of the neurostimulation parameter selection circuit 1422 enables selection, modification, and implementation of a particular set of parameters or settings for neurostimulation programming. The particular set of parameters or settings that are selected, modified, or implemented can be based on the parameters, such as described and depicted with reference to the in FIGS. 1-11.

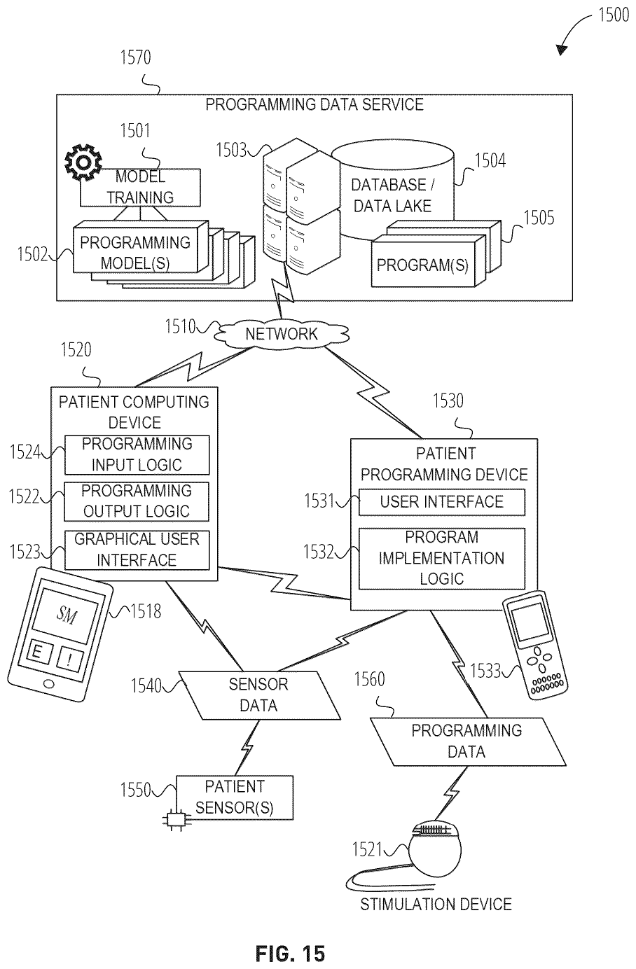

The external telemetry circuit 1440 provides the programming system 1402 (e.g., a closed-loop programming system) with wireless communication to and from another controllable device such as the implantable stimulator including transmitting one or a plurality of stimulation parameters (e.g., selected, identified, or modified stimulation parameters of a selected program) to the implantable stimulator, via programming data 1560 as described and depicted in connection with FIG. 15. In one embodiment, the external telemetry circuit 1440 also transmits power to the stimulator, such as implantable stimulator or stimulation device 1521 as described and depicted in connection with FIG. 15, through inductive coupling or the like.

The external communication device 1418 can provide a mechanism to conduct communications with a programming information source, such as a data service, program modeling system, to receive program information, settings and values, models, functionality controls, or the like, via an external communication link (not shown). In a specific example, the external communication device 1418 communicates with the data analysis system 1450 (e.g., such as a computing system) to obtain commands or instructions in connection with parameters or settings that are selected, modified, or implemented based on similarity metrics analysis from the data analysis system 1450. The external communication device 1418 may communicate using any number of wired or wireless communication mechanisms described in this document, including but not limited to IEEE 802.11 (Wi-Fi), Bluetooth, Infrared, and like standardized and proprietary wireless communications implementations. Although the external telemetry circuit 1440 and the external communication device 1418 are depicted as separate components within the programming system 1402, the functionality of both of these components can be integrated into a single communication chipset, circuitry, device, or the like.

The external storage device 1416 stores a plurality of existing neurostimulation waveforms, including definable waveforms for use as a portion of the pattern of the neurostimulation pulses, settings and setting values, other portions of a program, and related treatment efficacy indication values. In various embodiments, each waveform of the plurality of individually definable waveforms includes one or more pulses of the neurostimulation pulses and may include one or more other waveforms of the plurality of individually definable waveforms. Examples of such waveforms include pulses, pulse blocks, pulse trains, and train groupings, and programs. The existing waveforms stored in the external storage device 1416 can be definable at least in part by one or more parameters including, but not limited to the following: amplitude, pulse width, frequency, duration(s), electrode configurations, total charge injected per unit time, cycling (e.g., on/off time), waveform shapes, spatial locations of waveform shapes, pulse shapes, number of phases, phase order, interphase time, charge balance, and ramping.

The external storage device 1416 may also store a plurality of individually definable fields that can be implemented as part of a program. Each waveform of the plurality of individually definable waveforms is associated with one or more fields of the plurality of individually definable fields. Each field of the plurality of individually definable fields is defined by one or more electrodes of the plurality of electrodes through which a pulse of the neurostimulation pulses is delivered and a current distribution of the pulse over the one or more electrodes. A variety of settings in a program can be correlated to the control of these waveforms and definable fields.

The programming control circuit 1420 represents an embodiment of a control circuit and can translate or generate the specific stimulation parameters or changes which are to be transmitted to the implantable stimulator 1421, based on the results of the neurostimulation parameter selection circuit 1422. The pattern can be defined using one or more waveforms selected from the plurality of individually definable waveforms (e.g., defined by a program) stored in an external storage device 1416. In various embodiments, the programming control circuit 1420 checks values of the plurality of stimulation parameters against safety rules to limit these values within constraints of the safety rules. In one embodiment, the safety rules are heuristic rules.

The user interface 1410 represents an embodiment of user interface devices and allows the user (e.g., a patient, representative, clinician, etc.) to provide input relevant to therapy objectives, such as to switch programs or change operational use of the programs. The user interface 1410 includes a display screen 1412, a user input device 1414, and can implement or couple to the neurostimulation parameter selection circuit 1422, or data provided from the data analysis system 1450. The display screen 1412 can include any type of interactive or non-interactive screens, and the user input device 1414 can include any type of user input devices that supports the various functions discussed in this document, such as a touchscreen, keyboard, keypad, touchpad, trackball, joystick, mouse, physical human interaction (e.g., fingers, voice, sound, etc.), or the like. The user interface 1410 may also allow the user to perform other functions where user interface input is suitable (e.g., to select, modify, enable, disable, activate, schedule, or otherwise define a program, sets of programs, provide feedback, or input values, or perform other monitoring and programming tasks). Although not shown, the user interface 1410 may also generate a visualization of such characteristics of device implementation or programming and receive and implement commands to implement or revert the program and the neurostimulator operational values (including a status of implementation for such operational values). These commands and visualization can be performed in a review and guidance mode, status mode, or in a real-time programming mode.

The controller 1430 can be a microprocessor that communicates with the external telemetry circuit 1440, the external communication device 1418, the external storage device 1416, the programming control circuit 1420, the parameter selection circuit, and the user interface 1410 device, via a bidirectional data bus or the like. The controller 1430 can be implemented by other types of logic circuitry (e.g., discrete components or programmable logic arrays) using a state machine type of design. As used in this disclosure, the term “circuitry” should be taken to refer to discrete logic circuitry, firmware, to the programming of a microprocessor, or a combination thereof.

The data analysis system 1450 is configured to operate treatment action circuitry 1460, which can produce or initiate certain actions on the basis of device data (received and processed by device data processing circuit 1452) and input (received and processed by processing circuit 1454). The treatment action circuitry 1460 can identify one or more actions related to the neurostimulation treatment and provide outputs to a patient or a clinician using patient output circuitry 1462 or clinician (or other non-patient person, entity, program, or combination thereof) output circuitry 1464, respectively. Such outputs and actions provided by the outputs are based on the evaluation and detection of particular patient data, triage data, parameter data, device states from text and associated device data, machine learning models, similarity metrics, stimulation settings, stimulation programming parameters, or a combination thereof.

The data analysis system 1450 also is depicted as including a storage device 1456 to store or persist data related to the device data, text input, patient, clinician output, and related settings, logic, or algorithms. Other hardware features of the data analysis system 1450 are not depicted for simplicity but are suggested from functional capabilities and operations in the following figures.

As will be understood, patients who are experiencing chronic pain are often willing to provide detailed information regarding their current medical state within varying input options (e.g., freeform text answers to questions, questionnaires, question trees, associated applications, adaptation device data, etc.). For example, freeform text in the form of a narrative, explanatory statement, or interjection is easy for patients to produce, and can provide many details regarding a patient's actions, physiological, physical, and psychological state, prior historical events, and can reflect both objective and subjective results of neurostimulation treatment. Freeform text, however, can be time-consuming or difficult for physicians and clinicians to interpret, especially when patient feedback can be contradictory (e.g., “I felt good in the morning but was unable to do any activity”) or is incomplete without additional context (e.g., “I was unable to get out of bed”). Capturing patient feedback with the present systems can provide many new data points for treatment outcomes, can triage patients using alert notifications, and provide a basis for determining whether or why a particular neurostimulation treatment (and treatment program, programming value, programming effect) is or is not effective (e.g., beneficial clinical effects, detrimental clinical effects, etc.).

FIG. 15 illustrates a block diagram 1500, by way of example, an embodiment of data interactions among a neurostimulation system implemented as a programming data service 1570, for operation of a stimulation device 1521 with use of closed loop programming, open loop programming, partially closed loop programming, or a combination thereof. At a high level, a programming data service 1570 uses a trained model (one of programming models 1502) to generate programming settings and parameters (e.g., in one or more programs 1505) that are customized to the patient. The programming data service 1570 includes computer hardware 1503 to control the model training 1501 and other data processing operations, such as to generate or control diagnostic actions, alerts, programming recommendations, or programming actions. The programming settings and parameters may be implemented automatically or manually on the stimulation device 1521 (e.g., using the programming techniques referenced above) or via a patient computing device 1520 or patient programming device 1530.

The programming data service 1570 communicates with one or both of the patient computing and programming devices 1520, 1530 via a network 1510, to obtain training data for use by model training logic 1501. The training data may be stored in a database 1504 or another large-scale data store (e.g., data lake) for the patient or a population of patients. The programming data service 1570 may also include data analysis or processing engines (not shown) that parse and determine one or more similarity metrics of a particular patient from various inputs and correlated program usage (e.g., to determine what programs and programming settings are beneficial or detrimental to the patient). In some examples, the state of treatment may be based on correlating the historical use of a neurostimulation program or set of parameters with the current similarity metric of a patient (e.g., identifying that a pain condition became worse or better after beginning use of a particular program or location of a lead).

The programming data service 1570 can also analyze a variety of forms of patient input and patient data related to usage of a neurostimulation program or neurostimulation programming parameters. For instance, the programming data service 1570 can receive information from program usage, questionnaire selections, text input originating from a human patient, stimulation parameters, voxel intensities, imaging data, native space imaging data, region of interest, information from the structural components of the neuroanatomical areas relevant to the DBS, structural features, and the like via the patient computing and programming devices 1520, 1530. In addition to providing recommended programs, the programming data service 1570 can also provide therapy content, stimulation settings, parameter values, similarity metrics, regions of interest, brain objects, voxels in an ROI, or other recommendations to the patient computing and programming devices 1520, 1530.

A patient can provide training data (e.g., input data, evoked responses to similarity metrics, etc.) via the patient computing device 1520 using a user device 1518 or the patient programming device 1530 using a remote control 1533. Additional detail of how input data is collected is discussed with reference to the data processing logic and user interfaces discussed in related patent application. In an example, the patient computing device 1520 is a computing device (e.g., personal computer, tablet, smartphone, sensor, etc.) or other form of user-interactive device that receives and provides interaction with a patient using a graphical user interface 1523, with use of programming input logic 1524 and programming output logic 1522. For instance, the programming input logic 1524 can receive input from a patient via questionnaires, surveys, messages, sensors, or other inputs. The inputs may provide text related to pain or overall health, which can be used to identify a psychological state(s), physical state(s), physiological state(s), somatic state(s), or a combination of states of the patient, neurostimulation treatment results, or related conditions. As used herein, the terms “neurostimulator,” “stimulator,” “neurostimulation,” and “stimulation” generally refer to the delivery of electrical energy that affects the neuronal activity of neural tissue, which may be excitatory or inhibitory; for example, by initiating an action potential, inhibiting or blocking the propagation of action potentials, affecting changes in neurotransmitter/neuromodulator release or uptake, and inducing changes in neuro-plasticity or neurogenesis of tissue. It will be understood that other clinical effects and physiological mechanisms may also be provided through use of such stimulation techniques.

A patient programming device 1530 is depicted as including a user interface 1531 and program implementation logic 1532. The program implementation logic 1532 specifically can provide the patient with the ability to implement or switch to particular programs generated by programming data service 1570. Other forms of programming can also include the receipt of instructions, recommendations, or feedback (including clinician recommendations, behavioral modifications, etc., selected for the patient) that are automatically selected based on detected conditions.

The programming data service 1570 can also utilize sensor data 1540 from one or more patient sensors 1550 (e.g., wearables, sleep trackers, motion tracker, implantable devices, etc.) among one or more internal or external devices. The sensor data 1540 can be used to determine customized and current parameterizing techniques for therapy and multi-sensor paresthesia therapy or neurostimulation treatment results. In various examples, the stimulation device 1521 also includes sensors that contribute to the sensor data 1540 to be evaluated by the programming data service 1570.

In an example, the patient sensors 1550 are physical, physiological, biopsychosocial, or similar sensors that collect data relevant to physical, biopsychosocial (e.g., stress and/or mood biomarkers), or physiological factors relevant to stimulation settings based on similarity metrics and/or parameter values of a plurality of patients. Examples of such sensors might include a sleep sensor to sense the patient's sleep state (e.g., for detecting lack of sleep), a respiration sensor to measure patient breathing rate or capacity, a movement sensor to identify an amount or type of movement, a heart rate sensor to sense the patient's heart rate, a blood pressure sensor to sense the patient's blood pressure, an electrodermal activity (EDA) sensor to sense the patient's EDA (e.g., galvanic skin response), a facial recognition sensor to sense the patient's facial expression, a voice sensor (e.g., microphone) to sense the patient's voice, and/or an electrochemical sensor to sense stress biomarkers from the patient's body fluids (e.g., enzymes and/or ions, such as lactate or cortisol from saliva or sweat). Other types or form factors of sensor devices may also be utilized.

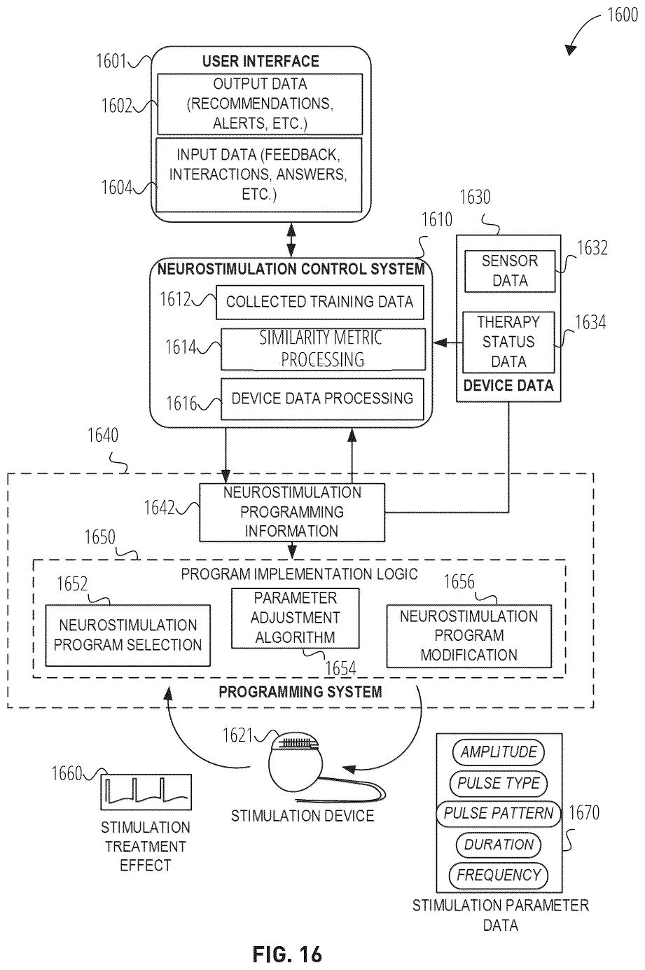

FIG. 16 illustrates, by way of example, an embodiment of a data processing flow 1600 affecting the neurostimulation treatment of a patient, including a neurostimulation control system 1610 based on collected training data 1612, similarity metric processing 1614, and device data processing 1616 functions. Here, additional details are provided on the data flow between the neurostimulation system 800 and an example user interface 1601. Other user interfaces and actions are not depicted for simplicity.

In this example, input data 1604 (e.g., parameter input, questionnaire answers, question trees, freeform text, patient feedback, interaction results, etc.) is obtained by the programming system 804. FIG. 16 also depicts the evaluation of device data 1630, such as sensor data 1632, therapy status data 1634, and other treatment aspects that can be obtained or derived from the neurostimulation device or related neurostimulation programming. Also in this example, output data 1602 (e.g., content) is obtained from the implantable device 802 at a user interface, such as in the form of recommendations, alerts, triage messages, imputation suggestions, treatment updates, patient information, and the like. The implantable device 802 can separately provide stimulation settings, parameter values, similarity metrics, regions of interest, brain objects, voxels in an ROI, or other recommendations to the clinician, doctor, user, software, or device representative separately from the patient.

The remainder of the data processing flow 1600 illustrates how data processing results from the implantable device 802 can be used to effect programming, such as in a closed loop (or partially-closed-loop, open-loop, or other) system. A programming system 1640 can use parameters or programming information 1642 (e.g., program neurostimulation programming information) provided from the implantable device 802 as an input to program implementation logic 1650. The program implementation logic 1650 can be implemented by a parameter adjustment algorithm 1654, which affects a neurostimulation program selection 1652 or a neurostimulation program modification 1656. For instance, some parameter changes can be implemented by a simple modification to a program operation; other parameter changes may require a new program to be deployed. The results of the parameter or program changes or selection results in definition or adjustment to various stimulation parameters at the neurostimulation stimulation device 1621, causing a different or new stimulation treatment effect 1660.

By way of example, stimulation parameter data 1670 includes operational parameters of the neurostimulation device, which are generated, identified, and/or evaluated by the present systems and techniques can include amplitude, frequency, duration, pulse width, pulse type, patterns of neurostimulation pulses, waveforms in the patterns of pulses, and like settings with respect to the intensity, type, and location of neurostimulator output on individual or a plurality of respective leads. The neurostimulator may use current or voltage sources to provide the neurostimulator output and apply any number of control techniques to modify the electrical simulation applied to anatomical sites or systems related to pain or analgesic effect.

In various embodiments, a neurostimulator program can be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

FIG. 17 is a flowchart depicting machine-learning pipeline 1700, according to some examples. The machine-learning pipeline 1700 may be used to generate a trained model, for example the trained machine-learning program 1802 of FIG. 18, to perform operations associated with searches and query responses.

Broadly, machine learning may involve using computer algorithms to automatically learn patterns and relationships in data, potentially without the need for explicit programming. Machine learning algorithms can be divided into three main categories: supervised learning, unsupervised learning, and reinforcement learning.

Supervised learning involves training a model using labeled data to predict an output for new, unseen inputs. Examples of supervised learning algorithms include linear regression, decision trees, and neural networks. Unsupervised learning involves training a model on unlabeled data to find hidden patterns and relationships in the data. Examples of unsupervised learning algorithms include clustering, principal component analysis, and generative models like autoencoders. Reinforcement learning involves training a model to make decisions in a dynamic environment by receiving feedback in the form of rewards or penalties. Examples of reinforcement learning algorithms include Q-learning and policy gradient methods.

Examples of specific machine learning algorithms that may be deployed, according to some examples, include logistic regression, which is a type of supervised learning algorithm used for binary classification tasks. Logistic regression models the probability of a binary response variable based on one or more predictor variables. Another example type of machine learning algorithm is Naïve Bayes, which is another supervised learning algorithm used for classification tasks. Naïve Bayes is based on Bayes' theorem and assumes that the predictor variables are independent of each other. Random Forest is another type of supervised learning algorithm used for classification, regression, and other tasks. Random Forest builds a collection of decision trees and combines their outputs to make predictions. Further examples include neural networks, which consist of interconnected layers of nodes (or neurons) that process information and make predictions based on the input data. Matrix factorization is another type of machine learning algorithm used for recommender systems and other tasks. Matrix factorization decomposes a matrix into two or more matrices to uncover hidden patterns or relationships in the data. Support Vector Machines (SVM) are a type of supervised learning algorithm used for classification, regression, and other tasks. SVM finds a hyperplane that separates the different classes in the data. Other types of machine learning algorithms include decision trees, k-nearest neighbors, clustering algorithms, and deep learning algorithms such as convolutional neural networks (CNN), recurrent neural networks (RNN), and transformer models. The choice of algorithm depends on the nature of the data, the complexity of the problem, and the performance requirements of the application.

The performance of machine learning models is typically evaluated on a separate test set of data that was not used during training to ensure that the model can generalize to new, unseen data. Although several specific examples of machine learning algorithms are discussed herein, the principles discussed herein can be applied to other machine learning algorithms as well. Deep learning algorithms such as convolutional neural networks, recurrent neural networks, and transformers, as well as more traditional machine learning algorithms like decision trees, random forests, and gradient boosting may be used in various machine learning applications.

Two example types of problems in machine learning are classification problems and regression problems. Classification problems, also referred to as categorization problems, aim at classifying items into one of several category values (for example, is this object an apple or an orange?). Regression algorithms aim at quantifying some items (for example, by providing a value that is a real number).

From the training phases 1804 of FIG. 18, generating a trained machine-learning program 1802 may include multiple phases that form part of the machine-learning pipeline 1700, including for example the following phases illustrated in FIG. 17:

Data collection and preprocessing 1702: This phase may include acquiring and cleaning data to ensure that it is suitable for use in the machine learning model. This phase may also include removing duplicates, handling missing values, and converting data into a suitable format.

Feature engineering 1704: This phase may include selecting and transforming the training data 1806 to create features that are useful for predicting the target variable. Feature engineering may include (1) receiving features 1808 (e.g., as structured or labeled data in supervised learning) and/or (2) identifying features 1808 (e.g., unstructured, or unlabeled data for unsupervised learning) in training data 1806.

Model selection and training 1706: This phase may include selecting an appropriate machine learning algorithm and training it on the preprocessed data. This phase may further involve splitting the data into training and testing sets, using cross-validation to evaluate the model, and tuning hyperparameters to improve performance.

Model evaluation 1708: This phase may include evaluating the performance of a trained model (e.g., the trained machine-learning program 1802) on a separate testing dataset. This phase can help determine if the model is overfitting or underfitting and determine whether the model is suitable for deployment.

Prediction 1710: This phase involves using a trained model (e.g., trained machine-learning program 1802) to generate predictions on new, unseen data.

Validation, refinement or retraining 1712: This phase may include updating a model based on feedback generated from the prediction phase, such as new data or user feedback.

Deployment 1714: This phase may include integrating the trained model (e.g., the trained machine-learning program 1802) into a more extensive system or application, such as a web service, mobile app, or IoT device. This phase can involve setting up APIs, building a user interface, and ensuring that the model is scalable and can handle large volumes of data.

FIG. 18 illustrates further details of two example phases, namely a training phase 1804 (e.g., part of the model selection and trainings 1706) and a prediction phase 1710 (part of prediction phase). Prior to the training phase 1804, feature engineering 1704 is used to identify features 1808. This may include identifying informative, discriminating, and independent features for effectively operating the trained machine-learning program 1802 in pattern recognition, classification, and regression. In some examples, the training data 1806 includes labeled data, known for pre-identified features 1808 and one or more outcomes. Each of the features 1808 may be a variable or attribute, such as an individual measurable property of a process, article, system, or phenomenon represented by a data set (e.g., the training data 1806). Features 1808 may also be of different types, such as numeric features, strings, and graphs, and may include one or more of content 1812, concepts 1814, attributes 1816, historical data 1818, and/or user data 1820, merely for example.

In training phase 1804, the machine-learning pipeline 1800 uses the training data 1806 to find correlations among the features 1808 that affect a predicted outcome or prediction/inference data 1822.

With the training data 1806 and the identified features 1808, the trained machine-learning program 1802 is trained during the training phase 1804 during machine-learning program training 1824. The machine-learning program training 1824 appraises values of the features 1808 as they correlate to the training data 1806. The result of the training is the trained machine-learning program 1802 (e.g., a trained or learned model).

Further, the training phase 1804 may involve machine learning, in which the training data 1806 is structured (e.g., labeled during preprocessing operations). The trained machine-learning program 1802 implements a neural network 1826 capable of performing, for example, classification and clustering operations. In other examples, the training phase 1804 may involve deep learning, in which the training data 1806 is unstructured, and the trained machine-learning program 1802 implements a deep neural network 1826 that can perform both feature extraction and classification/clustering operations.

In some examples, a neural network 1826 may be generated during the training phase 1804 and implemented within the trained machine-learning program 1802. The neural network 1826 includes a hierarchical (e.g., layered) organization of neurons, with each layer consisting of multiple neurons or nodes. Neurons in the input layer receive the input data, while neurons in the output layer produce the final output of the network. Between the input and output layers, there may be one or more hidden layers, each consisting of multiple neurons.

Each neuron in the neural network 1826 operationally computes a function, such as an activation function, which takes as input the weighted sum of the outputs of the neurons in the previous layer, as well as a bias term. The output of this function is then passed as input to the neurons in the next layer. If the output of the activation function exceeds a certain threshold, an output is communicated from that neuron (e.g., transmitting neuron) to a connected neuron (e.g., receiving neuron) in successive layers. The connections between neurons have associated weights, which define the influence of the input from a transmitting neuron to a receiving neuron. During the training phase, these weights are adjusted by the learning algorithm to optimize the performance of the network. Different types of neural networks may use different activation functions and learning algorithms, affecting their performance on different tasks. The layered organization of neurons and the use of activation functions and weights enable neural networks to model complex relationships between inputs and outputs, and to generalize to new inputs that were not seen during training.

In some examples, the neural network 1826 may also be one of several different types of neural networks, such as a single-layer feed-forward network, a Multilayer Perceptron (MLP), an Artificial Neural Network (ANN), a Recurrent Neural Network (RNN), a Long Short-Term Memory Network (LSTM), a Bidirectional Neural Network, a symmetrically connected neural network, a Deep Belief Network (DBN), a Convolutional Neural Network (CNN), a Generative Adversarial Network (GAN), an Autoencoder Neural Network (AE), a Restricted Boltzmann Machine (RBM), a Hopfield Network, a Self-Organizing Map (SOM), a Radial Basis Function Network (RBFN), a Spiking Neural Network (SNN), a Liquid State Machine (LSM), an Echo State Network (ESN), a Neural Turing Machine (NTM), DNN, or a Transformer Network, merely for example.

CNNs excel at finding predictive patterns in high-dimensional imaging data, while RNNs can model longitudinal disease progression by analyzing sequences of images over time. Autoencoders learn compressed representations of complex imaging data for efficient processing. Clustering algorithms group patients based on phenotypic imaging biomarkers in an unsupervised manner. Decision trees enable interpreting relationships and importance of variables determining optimal parameters. Random forests improve prediction accuracy and avoid overfitting. SVMs flexibly perform classification and regression. The choice of one or more machine learning algorithms depends on factors such as available data size and variety as well as the need for interpretability versus high performance. In general, deep neural networks are well-suited to learning from multimodal imaging data and automatically extracting biomarkers used to optimize deep brain stimulation therapy.

In addition to the training phase 1804, a validation phase may be performed on a separate dataset known as the validation dataset. The validation dataset is used to tune the hyperparameters of a model, such as the learning rate and the regularization parameter. The hyperparameters are adjusted to improve the model's performance on the validation dataset.

Once a model is fully trained and validated, in a testing phase, the model may be tested on a new dataset. The testing dataset is used to evaluate the model's performance and ensure that the model has not overfitted the training data.

In prediction phase 1810, the trained machine-learning program 1802 uses the features 1808 for analyzing query data 1828 to generate inferences, outcomes, or predictions, as examples of a prediction/inference data 1822. For example, during prediction phase 1810, the trained machine-learning program 1802 generates an output. Query data 1828 is provided as an input to the trained machine-learning program 1802, and the trained machine-learning program 1802 generates the prediction/inference data 1822 as output, responsive to receipt of the query data 1828.

In some examples, the trained machine-learning program 1802 may be a generative AI model. Generative AI is a term that may refer to any type of artificial intelligence that can create new content from training data 1806. For example, generative AI can produce text, images, video, audio, code, or synthetic data similar to the original data but not identical.

Some of the techniques that may be used in generative AI are:

Convolutional Neural Networks (CNNs): CNNs may be used for image recognition and computer vision tasks. CNNs may, for example, be designed to extract features from images by using filters or kernels that scan the input image and highlight important patterns.

Recurrent Neural Networks (RNNs): RNNs may be used for processing sequential data, such as speech, text, and time series data, for example. RNNs employ feedback loops that allow them to capture temporal dependencies and remember past inputs.

Generative adversarial networks (GANs): GNNs may include two neural networks: a generator and a discriminator. The generator network attempts to create realistic content that can “fool” the discriminator network, while the discriminator network attempts to distinguish between real and fake content. The generator and discriminator networks compete with each other and improve over time.

Variational autoencoders (VAEs): VAEs may encode input data into a latent space (e.g., a compressed representation) and then decode it back into output data. The latent space can be manipulated to generate new variations of the output data. VAEs may use self-attention mechanisms to process input data, allowing them to handle long text sequences and capture complex dependencies.

Transformer models: Transformer models may use attention mechanisms to learn the relationships between different parts of input data (such as words or pixels) and generate output data based on these relationships. Transformer models can handle sequential data, such as text or speech, as well as non-sequential data, such as images or code.

In generative AI examples, the output prediction/inference data 1822 include predictions, translations, summaries, or media content.

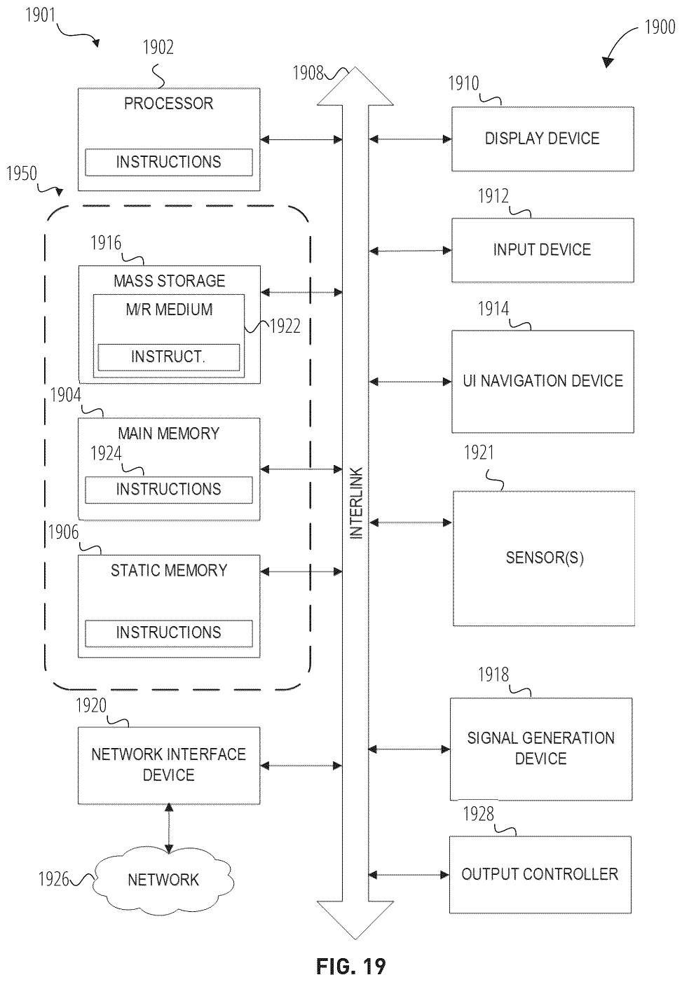

FIG. 19 is a block diagram 1900 illustrating a machine in the example form of a computer system 1901, within which a set or sequence of instructions can be executed to cause the machine to perform any one of the methodologies discussed herein, according to an example embodiment. In alternative embodiments, the machine operates as a standalone device or can be connected (e.g., networked) to other machines.

In a networked deployment, the machine may operate in the capacity of either a server or a client machine in server-client network environments, or it may function as a peer machine in peer-to-peer (or distributed) network environments. The machine can be a personal computer (PC), a tablet PC, a hybrid tablet, a personal digital assistant (PDA), a mobile telephone, an implantable pulse generator (IPG), an external trial stimulator (ETS), an external remote control (RC), a User's Programmer (UP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1901 includes at least one processor 1902 (e.g., a central processing unit (CPU), a graphics processing unit (GPU), a tensor processing unit (TPU) or combination thereof, processor cores, compute nodes, etc.), a main memory 1904 and a static memory 1906, which communicate with each other via a link 1908 (e.g., bus, interlink, etc.). The computer system 1901 can further include a video display unit 1910, an alphanumeric input device 1912 (e.g., a keyboard), and a user interface (UI) navigation device 1914 (e.g., a mouse). In one embodiment, the video display unit 1910, input device 1912, and/or user interface (UI) navigation device 1914 are incorporated into a touch screen or interactive display. The computer system 1901 can additionally include a storage device 1916 (e.g., a drive unit), a signal generation device 1918 (e.g., a speaker), an output controller 1928, a network interface device 1920, and one or more sensors 1921, such as a global positioning system (GPS) sensor, compass, accelerometer, or another sensor. It will be understood that other forms of machines or apparatuses (e.g., PIG, RC, CP devices, and the like) that are capable of implementing the methodologies discussed in this disclosure may not incorporate or utilize every component depicted in FIG. 19 (e.g., a GPU, video display unit, keyboard, etc.).

The storage device 1950 includes a mass storage 1916, a machine-readable medium 1922 on which is stored one or more sets of data structures and instructions 1924 (e.g., software) embodying or utilized by any one or more of the methodologies or functions described herein. The instructions 1924 can also reside, completely or at least partially, within the main memory 1904, static memory 1906, and/or within the processor 1902 during execution thereof by the computer system 1901, with the main memory 1904, static memory 1906, and the processor 1902 also constituting machine-readable media.

While the machine-readable medium 1922 is illustrated in an example embodiment to be a single medium, the term “machine-readable medium” can include a single medium or multiple media (e.g., a centralized or distributed database, and/or associated caches and servers) that store the one or more instructions 1924. The term “machine-readable medium” shall also be taken to include any tangible (e.g., non-transitory) medium that is capable of storing, encoding, or carrying instructions for execution by the machine and that cause the machine to perform any one or more of the methodologies of the present disclosure or that is capable of storing, encoding, or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1924 can further be transmitted or received over a communications network 1926 using a transmission medium via the network interface device 1920 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A, 5G, or other generational networks). The term “transmission medium” shall be taken to include any intangible medium that is capable of storing, encoding, or carrying instructions for execution by the machine, and includes digital or analog communications signals or other intangible medium to facilitate communication of such software.

As used herein, the terms “machine-storage medium,” “device-storage medium,” “machine-readable medium,” and “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media, and/or device-storage media include non-volatile memory, including by way of example semiconductor memory devices, e.g., erasable programmable read-only memory (EPROM), electrically erasable programmable read-only memory (EEPROM), field-programmable gate arrays (FPGAs), and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

The terms “transmission medium” and “signal medium” mean the same thing and may be used interchangeably in this disclosure. The terms “transmission medium” and “signal medium” shall be taken to include any intangible medium that is capable of storing, encoding, or carrying the instructions for execution by the machine depicted in block diagram 1900, and include digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a manner as to encode information in the signal.

The terms “machine-readable medium,” “computer-readable medium,” and “device-readable medium” mean the same thing and may be used interchangeably in this disclosure. The terms are defined to include both machine-storage media and transmission media. Thus, the terms include both storage devices/media and carrier waves/modulated data signals.

The above detailed description is intended to be illustrative, and not restrictive. The scope of the disclosure should, therefore, be determined with references to the appended claims, along with the full scope of equivalents to which such claims are entitled. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method for machine-learning based automated neurostimulation programming, the method comprising:
generating a database of previously tested stimulation settings and their clinical effect;
identifying a region of interest surrounding at least one lead;
determining a stimulation setting corresponding to one or more specific clinical effects associated with the region of interest, the one or more specific clinical effects including at least one beneficial clinical effect or at least one detrimental clinical effect;
employing a trained machine-learning model to identify a stimulation parameter value for a stimulation setting to set in a patient of interest based on the one or more specific clinical effects, the trained machine-learning model configured to classify the stimulation parameter value for the stimulation setting based at least in part on raw imaging data; and
generating an output providing the stimulation parameter value for the stimulation setting for the patient of interest.

2. The method of claim 1, further comprising:
determining a similarity metric between the patient of interest and a plurality of patients according to the region of interest.

3. The method of claim 2, further comprising:
using a single similarity metric or multiple similarity metrics; and
clustering stimulation settings from similar patients identified among the plurality of patients according to the region of interest.

4. The method of claim 1, further comprising:
suggesting or avoiding the stimulation settings based on cluster features identified among the plurality of patients according to the region of interest.

5. The method of claim 1, further comprising:
identify one or more commonalities between the patient of interest and the plurality of patients in the region of interest around the lead.

6. The method of claim 5, further comprising:
employing the trained machine-learning model to identify the one or more commonalities based at least in part on additional data types beyond raw imaging data.

7. The method of claim 6, wherein the additional data types beyond raw imaging data include at least one of disease state, comorbidities, patient states, or clinician evaluation.

8. The method of claim 1, further comprising:
identifying the stimulation parameter value for the stimulation setting to set in the patient of interest based on data-drive phenotypes.

9. The method of claim 1, further comprising:
identifying the stimulation parameter value for the stimulation setting to set in the patient of interest based on a hierarchical organization.

10. The method of claim 9, wherein the hierarchical organization includes a first level providing a best contact, a second level providing a best contact fraction, and a third level providing a best amplitude.

11. The method of claim 1, further comprising:
employing the trained machine-learning model to confirm that the model-identified stimulation parameter value for the stimulation setting to set in the patient of interest based on the one or more specific clinical effects matches known target settings for a patient.

12. The method of claim 11, wherein the confirmation is performed through use of at least one of computational modeling, imaging, clinical evaluation, symptomology scoring, or invasive testing.

13. The method of claim 1, further comprising:
employing the trained machine-learning model to determine target similarity metrics and thresholds for grouping patients by anatomical phenotypes.

14. The method of claim 13, wherein grouping patients by the anatomical phenotypes includes using different similarity metric correlations, varying the thresholds to evaluate accuracy of predictions, identifying the thresholds that maximize accuracy, compute different similarity metrics on various image features, or selecting the various image features that produce distinct patient groupings.

15. The method of claim 1, further comprising:
validating, by the trained machine-learning models, a particular similarity metric and threshold by testing prediction accuracy on a new patient.

16. A machine-storage medium embodying instructions that, when executed by a machine, cause the machine to perform operations comprising:
generating a database of previously tested stimulation settings and their clinical effect;
identifying a region of interest surrounding at least one lead;
determining a stimulation setting corresponding to one or more specific clinical effects associated with the region of interest, the one or more specific clinical effects including at least one beneficial clinical effect or at least one detrimental clinical effect;
employing a trained machine-learning model to identify a stimulation parameter value for a stimulation setting to set in a patient of interest based on the one or more specific clinical effects, the trained machine-learning model configured to classify the stimulation parameter value for the stimulation setting based at least in part on raw imaging data; and
generating an output providing the stimulation parameter value for the stimulation setting for the patient of interest.

17. The machine-storage medium embodying instructions of claim 16, further comprising:
determining a similarity metric between the patient of interest and a plurality of patients according to the region of interest;
using a single similarity metric or multiple similarity metrics; and
clustering stimulation settings from similar patients identified among the plurality of patients according to the region of interest.

18. The machine-storage medium embodying instructions of claim 16, further comprising:
identify one or more commonalities between the patient of interest and the plurality of patients in the region of interest around the lead; and
employing the trained machine-learning model to identify the one or more commonalities based at least in part on additional data types beyond raw imaging data, wherein the additional data types beyond raw imaging data include at least one of disease state, comorbidities, patient states, or clinician evaluation.

19. The machine-storage medium embodying instructions of claim 16, further comprising:
identifying the stimulation parameter value for the stimulation setting to set in the patient of interest based on data-drive phenotypes; and
identifying the stimulation parameter value for the stimulation setting to set in the patient of interest based on a hierarchical organization.

20. A system for machine-learning based automated neurostimulation programming, the system comprising:
one or more processors; and
one or more memory storing instructions, which when executed by the one or more processors, cause the one or more processors to perform operations that:
generate a database of previously tested stimulation settings and their clinical effect;
identify a region of interest surrounding at least one lead;
determine a stimulation setting corresponding to one or more specific clinical effects associated with the region of interest, the one or more specific clinical effects including at least one beneficial clinical effect or at least one detrimental clinical effect;
employ a trained machine-learning model to identify a stimulation parameter value for a stimulation setting to set in a patient of interest based on the one or more specific clinical effects, the trained machine-learning model configured to classify the stimulation parameter value for the stimulation setting based at least in part on raw imaging data; and
generate an output providing the stimulation parameter value for the stimulation setting for the patient of interest.
